(19) United States
(12) Patent Application Publication
(10) Pub. No.: US 2024/0139197 A1
(43) Pub. Date: May 2, 2024

(54) INHIBITION OF KDM5A FOR PROMOTING ANTIGEN PRESENTATION, INCREASING CD8+ T CELL INFILTRATION AND BOOSTING ANTI-TUMOR IMMUNE RESPONSE

(71) Applicant: The Wistar Institute of Anatomy and Biology, Philadelphia, PA (US)

(72) Inventor: Rugang Zhang, Elkins Park, PA (US)

(21) Appl. No.: 18/483,886

(22) Filed: Oct. 10, 2023

Related U.S. Application Data
(60) Provisional application No. 63/378,895, filed on Oct. 10, 2022.

(51) Int. Cl. A61K 31/519; A61K 31/7105; A61K 38/21; A61P 35/00

(52) U.S. Cl. CPC A61K 31/519; A61K 31/7105; A61K 38/217; A61P 35/00

(57) ABSTRACT

Methods of treating cancer, promoting antigen presentation, or increasing CD8+ T cell infiltration in tumors by inhibiting KDMA5 are provided.

### STATEMENT REGARDING FEDERALLY SPONSORED RESEARCH OR DEVELOPEMNT

This invention was made with government support under CA202919, CA239128, and CA228991 awarded by the National Institute of Health and OC180109 and OC19081 awarded by the Department of Defense. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “WST-192.US” (created Oct. 10, 2023, 2,877 bytes).

### BACKGROUND

Immune cell infiltration is major predictor of prognosis for multiple types of cancer, including epithelial ovarian cancer (EOC). Multiple studies have demonstrated that tumor genetic or epigenetic makeup may play a role in the regulation of intra-tumor immune infiltration and immune evasion. Given the reversible nature of epigenetic regulation, it is imperative to understand epigenetic basis of immune infiltration. These epigenetic regulators may be leveraged to develop novel therapeutic approaches that promote immune infiltration to boost anti-tumor immunity. Tumor antigen processing and presentation pathway is one of the key elements required for anti-tumor immune response. Expression of genes involved in the antigen processing and presentation pathway is often altered during tumor development and progression. Major histocompatibility class I (MHC class I) plays a key role in anti-tumor immune response by binding tumor intracellular peptides. Recognition of antigens presented in the context of MHC class I by professional antigen presenting cells results in activation or priming of CD8+ T cell response. MHC class I complex is not essential for cancer cell viability and, therefore, cancer cells often reduce expression of genes encoding MHC class I and other genes representing antigen presentation machinery to evade immune surveillance. However, the mechanisms by which cancer cells inhibit antigen processing and presentation to evade immune surveillance are poorly understood. Therefore, it is critically important to study the role of epigenetic regulators of antigen presentation pathway.

What is needed are therapies to improve anti-tumor immune responses.

### SUMMARY OF THE INVENTION

In one aspect, provided herein is a method of increasing intratumor CD8⁺ T-cell infiltration in a subject in need thereof, the method comprising administering a KDM5A inhibitor to the subject.

In another aspect, provided herein is a method of augmenting tumor antigen processing and/or presentation in a subject in need thereof, the method comprising administering a KDM5A inhibitor to the subject.

In yet another aspect, provided herein is a method of treating cancer in a subject in need thereof, the method comprising administering a KDM5A inhibitor and i) a cytotoxic agent, ii) interferon gamma, and/or iii) a checkpoint inhibitor to the subject.

In certain embodiments, the subject has ovarian cancer. In certain embodiments, the subject has high-grade serous ovarian cancer (HGSOC). In certain embodiments, the subject has triple negative breast cancer. In certain embodiments, the KDM5A inhibitor is CPI-455. In certain embodiments, the KDM5A inhibitor is administered in an amount ranging from about 0.01 mg to 10 mg.

Other aspects and advantages of the invention will be readily apparent from the following detailed description of the invention.

### BRIEF DESCRIPTION OF THE DRAWINGS

FIG. 1A-FIG. 1E show KDM5A is amplified in EOC and its amplification/overexpression negatively correlates with infiltration of CD8+ T cells. (FIG. 1A) Schematic of the experimental design for the identification of epigenetic factors regulating infiltration of CD8+ T cells in the TCGA HGSOC dataset. (FIG. 1B) Correlation between KDM5A and GZMB expression based on RNA-seq analysis in the TCGA HGSOC dataset. (FIG. 1C) Significant correlation between KDM5A and GZMB expression based on RNA-seq analysis from 24 TCGA cancer types with at least 100 patients. The arrow indicates HGSOC. (FIG. 1D) High KDM5A expression correlates with significantly lower levels of CD8+0 T cell infiltration in 124 cases of HGSOC. (FIG. 1E) Comparison of overall survival between HGSOC with or without KDM5A amplification/overexpression in the TCGA HGSOC dataset. Data represent mean±SEM; P values were calculated by using a two-tailed Student t test except in FIG. 1B by using Pearson r analysis, in FIG. 1C by using Fisher's exact test, and in FIG. 1E using log rank test.

FIG. 2A-FIG. 2J show KDM5A inhibition suppresses tumor growth in vivo in an immune-competent mouse model of ovarian cancer. (FIG. 2A) Expression of Kdm5a, H3K4me3, and a loading control β-actin in doxycycline-inducible shKdm5a ID8 mouse EOC cells treated with or without 2 μg/mL of doxycycline. (FIG. 2B) ID8 cells were plated into 24 well plates and treated with 2 μg/mL of doxycycline the same day. Cells were incubated for 8 days. Relative cell viability was determined by colony formation assay. (FIG. 2C) Expression of Kdm5a, H3K4me3, and a loading control β-actin in ID8 cells treated with 10uM CPI-455 or vehicle control. (FIG. 2D) ID8 cells were plated into 24 well plates and incubated with 10 μM CPI-455 for 8 days with fresh drug-containing media every 3 days. Relative cell viability was determined by colony formation assay. (FIG. 2E) Ascites produced in C57BL/6 mice injected with doxycycline-inducible shKdm5a ID8 cells treated with control or doxycycline containing food. (FIG. 2F) Quantification of ascites volumes from FIG. 2E. (FIG. 2G) Kaplan-Meier survival curves for the indicated groups. (FIG. 2H) Ascites produced in C57BL/6 mice injected with ID8 cells treated with vehicle control or KDM5A inhibitor CPI-455. (FIG. 21) Quantification of ascites volumes from FIG. 2H. (FIG. 2J) Kaplan-Meier survival curves for the indicated groups. Data represent mean±SEM of three biological repeats or 5 mice per group; P-value was calculated by using a two-tailed Student t test except in FIG. 2G and FIG. 2J by log rank tests.

FIG. 3A-FIG. 3C show CD8+ T cells mediate anti-tumor immune response induced by KDM5A inhibition. (FIG. 3A) Infiltration of CD8+ T cells or (FIG. 3B) active CD69+ CD8+ T cells in ascites collected from the indicated treatment groups were analyzed by flow cytometry at the end of experiment (n=5 mice per group). Data represent mean±SEM; P values were calculated using a two-tailed t test. (FIG. 3C) Kaplan-Meier survival curves for the indicated groups (n=5 mice per group). P value was calculated by log rank test.

FIG. 4A-FIG. 4M show KDM5A regulates antigen processing and presentation pathway. (FIG. 4A) Experimental strategy used to identify Kdm5a direct target genes. GSEA pathway analysis revealed that antigen processing and presentation pathway was enriched for the identified Kdm5a direct target genes (including H2-K1, H2-D1, and Tapbp). (FIG. 4B) CUT&RUN tracks of the newly identified Kdm5a target gene H2-K1 for Kdm5a and H3K4me3 in ID8 cells. (FIG. 4C) Expression of H2-K1 in doxycycline-inducible shKdm5a ID8 cells, Kdm5a inhibitor CPI-455, or Kdm5a knockout cells with or without IFNγ stimulation determined by RT-qPCR. (FIG. 4D) Cell surface expression of H2-K1 in control and Kdm5a knockout ID8 cells with or without IFNy stimulation was determined by FACS analysis. (FIG. 4E) Quantification of FIG. 4D. (FIG. 4F) Cell surface expression of H2-K1 in control and doxycyclineinducible Kdm5a-knockdown ID8 cells with or without IFNγ stimulation was determined by FACS analysis. (FIG. 4G) Quantification of FIG. 4F. (FIG. 4H) Cell surface expression of H2-K1 in D8 cells treated with vehicle or CPI-455 with or without IFNγ stimulation was determined by FACS analysis. (FIG. 4I) Quantification of FIG. 4H; An isotype matched IgG was used as a negative control for all the FACS analysis. (FIG. 4J-FIG. 4K) ID8 cells treated with or without CPI455, Kdm5a knockout or inducible Kdm5a knockdown were treated with IFNγ0 and then subjected to ChIP analysis using anti-H3K4me3 (FIG. 4J) or anti-KDM5A (FIG. 4K) antibodies. (FIG. 4L) Correlation between KDM5A and HLA-A or HLA-B expression based on RNA-seq analysis in the TCGA HGSOC dataset. (FIG. 4M) Significant correlation between KDM5A and HLA-A or HLA-B expression based on RNA-seq analysis from 24 TCGA cancer types with at least 100 patients. The arrows point to HGSOCs. Data represent mean±SEM of three biological repeats; P-value was calculated by using a two-tailed Student t test except in FIG. 4L by using Pearson r analysis test and FIG. 4M by using Fisher's exact test.

FIG. 5A-FIG. 5G show KDM5A is overexpressed in HGSOC and its expression negatively correlates with infiltration of CD8+ T cells. (FIG. 5A) Correlation between KDM5A and CD8A expression in the TCGA HGSOC dataset. (FIG. 5B) Correlation between KDM5A and CD8A expression in a published laser capture and microdissected (LCM) HGSOC dataset. (FIG. 5C) Correlation between KDM5A and PRFI expression in the TCGA HGSOC dataset. (FIG. 5D) Representative images of KDM5A IHC staining of HGSOC in a tumor microarray. (FIG. 5E) Relative expression of KDM5A in LCM of HGSOC and normal human ovarian surface epithelial cells (HOSE) or fallopian tube epithelial cells (FTE). (FIG. 5F) Expression of KDM5A and a loading control β-actin in the indicated FTE cells and HGSOC cell lines determined by immunoblot. (FIG. 5G) Dose response curves to KDM5A inhibitor CPI-455 determined by colony formation assay in the indicated HGSOC cell lines. Data represent mean±SEM; P values were calculated by using a two-tailed Student t test except in FIG. 5A and FIG. 5B by using Pearson r analysis.

FIG. 6A-FIG. 6K show KDM5A inhibition promotes anti-tumor immune response in vivo. (FIG. 6A) Expression of H3K4me3, Kdm5a and a loading control b-actin in an independent second doxycycline-inducible shKdm5a ID8 clone treated with or without 2 μg/mL of doxycycline. (FIG. 6B) Same as FIG. 6A, but doxycycline-inducible shKdm5a ID8 cells were plated into 24 well plates and treated with 2 μg/mL of doxycycline the same day. Cells were incubated for 8 days. Relative cell viability was determined by colony formation assay. (FIG. 6C) Expression of H3K4me3, Kdm5a and a loading control b-actin in HGS2 cells treated 10 μM of CPI-455 or vehicle controls for 72 hours. (FIG. 6D) Same as FIG. 6C, but HGS2 cells incubated with 10 μM CPI-455 for 8 days with fresh drug-containing media every 3 days. Relative cell viability was determined by colony formation assay. (FIG. 6E-FIG. 6F) Bioluminescence signal was measured by IVIS system in indicated animal groups in the immunocompetent C57BL/6 mouse model once a week (FIG. 6E) and the survival of the indicated groups was determined by Kaplan-Meier survival curves (FIG. 6F) (n=5 mice per group). (FIG. 6G-FIG. 6H) Ascites formed in C57BL/6 mice injected with an independent second doxycycline-inducible shKdm5a ID8 clone treated with or without doxycycline containing food (FIG. 6G). Ascites volumes produced from the indicated groups were quantified (FIG. 6H). n=5 mice per group. (FIG. 6I) Body weight of tumor bearing mice at the indicated time points. n=5 mice per group. (FIG. 6J-FIG. 6K) Bioluminescence signal was measured by IVIS system in indicated animal groups in the immunocompromised NSG mouse model once a week (FIG. 6J), or examined for Kaplan-Meier survival curves for the indicated groups (FIG. 6K). Data represent mean +SEM; P values were calculated by using a two-tailed Student t test except in FIG. 6H and FIG. 6K by log rank tests.

FIG. 7A-FIG. 7E show the effects of KDM5A inhibition on changes in tumor immune microenvironment. (FIG. 7A) The gating strategy used for determining the indicated cell populations. (FIG. 7B) Total number of CD8+ T cells in ascites collected from the indicated treatment groups were analyzed by flow cytometry (n=5 mice per group). (FIG. 7C) Percentages of PD1+ CD8+ T cells in ascites collected from the indicated treatment groups were analyzed by flow cytometry (n=5 mice per group). (FIG. 7D) Percentages of PD-L1+ ID8 cancer cells, PD-L1+ myeloid cells, and PD1+ CD4+T cells in ascites collected from the indicated treatment groups were analyzed by flow cytometry (n=5 mice per group). (FIG. 7E) Percentages of CD11b+ dendritic cells, myeloid cells (CD45+ CD11B+), CD4+ T cells, and B (CD45+ CD19+) cells in ascites collected from the indicated treatment groups were analyzed by flow cytometry (n=5 mice per group). Data represent mean±SEM; P values were calculated by using a two-tailed Student t test.

FIG. 8A-FIG. 8K show KDM5A regulates antigen processing and presentation pathway. (FIG. 8A) Validation of Kdm5a knockout and upregulation of H3K4me3 in Kdm5a knockout ID8 cells by immunoblot. β-actin expression was used as a loading control. (FIG. 8B) GSEA KEGG enrichment analysis of differentially expressed genes (DEGs) between control and Kdm5a knockout ID8 cells. NES, normalized enrichment score. (FIG. 8C) Enrichment plot of GSEA for antigen processing and presentation pathway. (FIG. 8D) Heatmap of the antigen processing and presentation pathway genes upregulated in Kdm5a-knockout ID8 cells. (FIG. 8E) Expression of Kdm5a and a loading control β-actin in inducible Kdm5a knockdown HGS2 cells with or without doxycycline treatment determined by immunoblot. (FIG. 8F) Cell surface expression of H2-K1 in HGS2 cells with or without inducible Kdm5a knockdown stimulated with IFNy was determined by FACS analysis. (FIG. 8G) Quantification of FIG. 8F. (FIG. 8H) Cell surface expression of H2-K1 in HGS2 cells treated with or without CPI455 and stimulated with IFNy was determined by FACS analysis. (FIG. 8I) Quantification of FIG. 8H; (FIG. 8J) Correlation between CD8A and HLA-A or HLA-B expression based on RNA-seq analysis in the TCGA HGSOC dataset. (FIG. 8K) Correlation between GZMB and HLA-A or HLA-B expression based on RNA-seq analysis in the TCGA HGSOC dataset. Data represent mean +SEM; P values were calculated by using a two-tailed Student t test except in FIG. 8J and FIG. 8K by using Pearson r analysis and FIG. 8B and FIG. 8C by using GSEA.

FIG. 9 provides tables showing the timelines for in vivo animal experiments.

FIG. 10 provides a table showing a list of identified epigenetic regulators whose expression negatively correlates with infiltration of CD8+ T cells and predicts a poor survival in HGSOC.

FIG. 11 provides a table showing the correlation between expression of KDM5A and GZMB in 24 TCGA datasets.

FIG. 12 provides a table showing the correlation between expression of KDM5A and HLA-A or HLA-B in 24 TCGA datasets.

### DETAILED DESCRITPION OF THE INVENTION

It is demonstrated herein that KDM5A represses expression of genes involved in tumor antigen processing and presentation. This results in decreased CD8+ T cells infiltration and ultimately inhibits anti-tumor immune response. KDM5A inhibition genetically or using small molecule inhibitors, restores expression of antigen presentation genes. Consistently, KDM5A inhibition reduces tumor burden and improves survival of tumor bearing mice in vivo in a CD8+ T cell-dependent manner in a syngeneic EOC mouse model. Thus, our studies establish KDM5A as an epigenetic therapeutic target whose inhibition boosts anti-tumor immune response.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

“Upregulate” and “upregulation”, as used herein, refer to an elevation in the level of expression of a product of one or more genes in a cell or the cells of a tissue or organ.

“Inhibit” or “downregulate”, as used herein refer to a reduction in the level of expression of a product of one or more genes in a cell or the cells of a tissue or organ.

By the general terms “blocker”, “inhibitor” or “antagonist” is meant an agent that inhibits, either partially or fully, the activity or production of a target molecule, e.g., as used herein, e.g., KDM5A. In particular, these terms refer to a composition or compound or agent capable of decreasing levels of gene expression, mRNA levels, protein levels or protein activity of the target molecule. Illustrative forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Illustrative non-limiting mechanisms of antagonist inhibition include repression of ligand synthesis and/or stability (e.g., using, antisense, ribozymes or RNAi compositions targeting the ligand gene/nucleic acid), blocking of binding of the ligand to its cognate receptor (e.g., using anti-ligand aptamers, antibodies or a soluble, decoy cognate receptor), repression of receptor synthesis and/or stability (e.g., using, antisense, ribozymes or RNAi compositions targeting the ligand receptor gene/nucleic acid), blocking of the binding of the receptor to its cognate receptor (e.g., using receptor antibodies) and blocking of the activation of the receptor by its cognate ligand (e.g., using receptor tyrosine kinase inhibitors). In addition, the blocker or inhibitor may directly or indirectly inhibit the target molecule.

The terms “RNA interference,” “RNAi,” “miRNA,” and “siRNA” refer to any method by which expression of a gene or gene product is decreased by introducing into a target cell one or more double-stranded RNAs, which are homologous to a gene of interest (particularly to the messenger RNA of the gene of interest). Gene therapy, i.e., the manipulation of RNA or DNA using recombinant technology and/or treating disease by introducing modified RNA or modified DNA into cells via a number of widely known and experimental vectors, recombinant viruses and CRISPR technologies, may also be employed in delivering, via modified RNA or modified DNA, effective inhibition of KDM5A pathways and gene products and β adrenergic pathways and gene products to accomplish the outcomes described herein with the combination therapies described. Such genetic manipulation can also employ gene editing techniques such as CRISPR (Clustered Regularly Interspaced Short Palindromic

Repeats) and TALEN (transcription activator-like effector genome modification), among others. See, for example, the textbook National Academies of Sciences, Engineering, and Medicine. 2017. Human Genome Editing: Science, Ethics, and Governance. Washington, DC: The National Academies Press. https://doi.org/10.17226/24623, incorporated by reference herein for details of such methods.

A “subject” is a mammal, e.g., a human, mouse, rat, guinea pig, dog, cat, horse, cow, pig, or non-human primate, such as a monkey, chimpanzee, baboon or gorilla. The term “patient” may be used interchangeably with the term subject. In one embodiment, the subject is a human. The subject may be of any age, as determined by the health care provider. In certain embodiments described herein, the patient is a subject who has previously been diagnosed with cancer. The subject may have been treated for cancer previously, or is currently being treated for cancer.

“Sample” as used herein means any biological fluid or tissue that contains blood cells, immune cells and/or cancer cells. In one embodiment, the sample is whole blood. In another embodiment, the sample is plasma. Other useful biological samples include, without limitation, peripheral blood mononuclear cells, plasma, saliva, urine, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. Such samples may further be diluted with saline, buffer or a physiologically acceptable diluent. Alternatively, such samples are concentrated by conventional means.

The term “cancer” or “proliferative disease” as used herein means any disease, condition, trait, genotype or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive and is also referred to as a neoplasm. Tumors can be either benign (not cancerous) or malignant. The methods described herein are useful for the treatment of cancer and tumor cells, i.e., both malignant and benign tumors. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multi-drug resistant cancers. In one embodiment, the cancer is lung cancer. In another embodiment, the cancer is ovarian cancer. In certain embodiments, the cancer is high-grade serous ovarian cancer (HGSOC). In other embodiments, the cancer is triple negative breast cancer. Triple-negative breast cancer (TNBC) is any breast cancer that lacks or show low levels of estrogen receptor (ER), progesterone receptor (PR) and human epidermal growth factor receptor 2 (HER2) overexpression and/or gene amplification.

“Control” or “control level” as used herein refers to the source of the reference value for KDM5A levels as well as the particular panel of control subjects identified in the examples below. In some embodiments, the control subject is a healthy subject with no disease. In another embodiment, the control subject is a patient who has been successfully treated for cancer. In yet other embodiments, the control or reference is the same subject from an earlier time point. Selection of the particular class of controls depends upon the use to which the diagnostic/monitoring methods and compositions are to be put by the physician.

The terms “analog”, “modification” and “derivative” refer to biologically active derivatives of the reference molecule that retain desired activity as described herein. Preferably, the analog, modification or derivative has at least the same desired activity as the native molecule, although not necessarily at the same level. The terms also encompass purposeful mutations that are made to the reference molecule.

By “fragment” is intended a molecule consisting of only a part of the intact full-length polypeptide sequence and structure. The fragment can include a C terminal deletion, an N terminal deletion, and/or an internal deletion of the native polypeptide. A fragment will generally include at least about 5-10 contiguous amino acid residues of the full length molecule, preferably at least about 15-25 contiguous amino acid residues of the full length molecule, and most preferably at least about 20 50 or more contiguous amino acid residues of the full length molecule, or any integer between 5 amino acids and the full length sequence, provided that the fragment in question retains the ability to elicit the desired biological response, although not necessarily at the same level.

By the term “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the framework antibody is an antibody fragment. The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding fragment” of an antibody. These antibody fragments are obtained using conventional techniques known to those in the art, and the fragments can be screened for utility in the same manner as whole antibodies. Antibody fragments include, without limitation, immunoglobulin fragments including, without limitation: single domain (Dab; e.g., single variable light or heavy chain domain), Fab, Fab′, F(ab′)2, and F(v); and fusions (e.g., via a linker) of these immunoglobulin fragments including, without limitation: scFv, scFv2, scFv-Fc, minibody, diabody, triabody, and tetrabody. The antibody may also be a protein (e.g., a fusion protein) comprising at least one antibody or antibody fragment.

The term “derived from” is used to identify the original source of a molecule (e.g., bovine or human) but is not meant to limit the method by which the molecule is made which can be, for example, by chemical synthesis or recombinant means.

As used herein, the term “a therapeutically effective amount” refers an amount sufficient to achieve the intended purpose. For example, an effective amount of a KDM5A inhibitor is sufficient to promoter antigen presentation, increase CD8+ T-cell infiltration, and/or boost anti-tumor immune response. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

The compositions described herein may be administered using any suitable route of administration. For example, compositions may be administered via intravenous, parenteral, subcutaneous, intramuscular, intracranial, intraorbital, ophthalmic, intraventricular, intracapsular, intraspinal, intracisteral, intraperitoneal, intranasal, or aerosol administration. The route of administration for each composition (e.g., KDM5A inhibitor, chemotherapeutic agents, checkpoint inhibitors) may be determined individually and may be the same or different.

The agent may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As used herein, “disease”, “disorder” and “condition” are used interchangeably, to indicate an abnormal state in a subject.

### KDM5A

In one embodiment, the method includes inhibiting or reducing KDM5A in the subject. Lysine-specific demethylase 5A (KDM5A), also known as Jumonji/ARID domaincontaining protein 1A (JARID1A) or retinoblastoma-binding protein 2 (RBP2), is an oncogene that is often amplified or overexpressed in several types of cancer (15,16). KDM5A is a demethylase that removes the lysine 4 trimethylation histone H3 (H3K4me3) epigenetic mark (17). H3K4me3 mark is present at the promoter sites of transcriptionally active genes (18,19). By removing H3K4me3, KDM5A represses multiple tumor suppressor genes involved in cell cycle (20), invasion (21), epithelial-mesenchymal transition (22), chemoresistance (16,23) and other processes beneficial for tumor progression (15). Several small-molecule KDM5A inhibitors have been developed and were shown to be effective in inhibiting KDM5A enzymatic activity as evidenced by upregulating H3K4me3 levels (24-26). However, despite its pharmacological efficacy in blocking KDM5A enzymatic activity, KDM5A inhibitors only demonstrated limited effectiveness at suppressing cancer cell viability as single agents in vitro (27). At the same time, there is evidence to suggest that KDM5A may exhibit its oncogenic activity through remodeling the tumor immune microenvironment (15). However, the role of KDM5A in controlling antigen presentation and the associated infiltration of CD8+ T cells was previously unclear.

The infiltration of effector CD8+ T cells into tumors is one of the major predictors of clinical outcome for epithelial ovarian cancer (EOC) patients. Immune cell infiltration is a complex process that could be affected by the epigenetic makeup of the tumor. It is demonstrated herein that a lysine 4 histone H3 (H3K4) demethylase KDM5A impairs immune cell infiltration and inhibits anti-tumor immune response. Mechanistically, KDM5A silences genes involved in antigen processing and presentation pathway. Antigen processing and presentation is a critical step that is required for CD8+ T cells infiltration and activation of CD8+ T cell mediated anti-tumor immune response. KDM5A inhibition restores the expression of antigen presentation pathway in vitro and promotes anti-tumor immune response mediated by CD8+ T cells in vivo in a syngeneic EOC mouse model. Notably, a negative correlation between expression of KDM5A and genes involved in antigen processing and presentation pathway such as HLA-A and HLA-B is observed in the majority of cancer types. In summary, the results described herein establish KDM5A as a regulator of CD8+ T cells tumor infiltration and demonstrate that KDM5A inhibition is a novel therapeutic strategy aiming to boost anti-tumor immune response.

In one embodiment, the method includes administering an effective amount of an inhibitor of KDM5A to a subject in need thereof. Inhibitors of KDM5A include, without limitation, the 3-azabicyclo(3.1.0)hexane derivatives disclosed in WO 2021/223699 A1, which is incorporated herein by reference. In certain embodiments, the inhibitor has a structure of Formula (I):

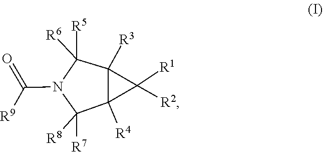

wherein R¹ represents Cyc1, —CO—Cyc2 or —CONR ¹⁰R¹¹;
Cyc1 represents a 5 to 9 membered aromatic hetero ring or 5 membered non-aromatic hetero ring, each of which may be substituted with 1 to 5 R¹²;
R¹² represents (1) C1-4 alkyl, (2) C3-7 cycloalkyl, (3) C1-4 haloalkyl, (4) C1-4 alkoxy, (5) phenyl which may be substituted with 1 to 3 R¹⁷, (6) C1-4 alkyl which is substituted with phenyl, (7) dimethylamino, (8) pyridyl or (9) 1-(cyclopropy lmethyl) pyrazol-3-yl;
a plurality of R¹² may be the same or different; two R¹² together with an atom to which these R¹² are attached may form a C3-5 cycloalkane, wherein the carbon atom of C3-5 cycloalkane may be replaced with hetero atom selected from 1 to 2 N, O and S;
R¹⁷ represents C1-4 alkyl, C1-4 alkoxy or halogen; a plurality of R¹⁷ may be the same or different;
Cyc2 represents a C3-12 mono or bicyclic carbocycle or a 5-to 9-membered mono or bicyclic heterocycle, each of which may be substituted with 1 to 5 R¹³;
R¹³ represents C1-4 alkyl, C1-4 alkoxy or halogen;
a plurality of R¹³ may be the same or different;
R¹⁰ represents

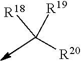

wherein R¹⁸ and R¹⁹ independently represents C1-4 alkyl;
R¹⁸ and R¹⁹ together with a carbon atom to which R¹⁸ and R¹⁹ arc attached may for m a C3-5 cycloalkane;
R²⁰ represents a hydrogen atom, C1-4 alkyl, C1-4 haloalkyl or nitrile;
(in the group, the arrow indicates the binding to the nitrogen atom of-CON<);
R¹¹ represents a hydrogen atom, C1-4 alkyl or 1 to 9 deuterated C1-4 alkyl;
R², R³, R⁴, R⁵, R⁶, R⁷ and R⁸ independently represent a hydrogen atom, C1-4 alkyl, halogen or C1-4 alkoxy;
R⁹ represents imidazole which may be substituted with 1 to 3 R¹⁴ or pyrazole which may be substituted with 1 to 3 R¹⁵;
R¹⁴ represents (1) C1-8 alkyl, (2) C3-7 cycloalkyl which may be substituted with C1-4 alkyl, (3) C1-8 haloalkyl, (4) C1-8 alkyl which is substituted with Cyc3 which may be substituted with 1 to 3 R¹⁶ or (5) C1-8 alkyl which is substituted with phenoxy;
Cyc3 represents phenyl, C3-7 cycloalkyl, pyridyl, thiazolyl or tetrahydropyranyl;
R¹⁶ represents C1-4 alkyl, halogen, C1-4 alkoxy or cyano;
a plurality of R¹⁴ may be the same or different;
a plurality of R¹⁶ may be the same or different;
R¹⁵ represents (1) C1-8 alkyl, (2) C3-7 cycloalkyl which may be substituted with C1-4 alkyl, (3) C1-8 haloalkyl, (4) C1-8 alkyl which is substituted with Cyc4 which may be substituted with 1 to 3 R²¹ or (5) C1-8 alkyl which is substituted with phenoxy;
Cyc4 represents phenyl, C3-7 cycloalkyl, pyridyl, thiazolyl or tetrahydropyranyl;
R²¹ represents C1-4 alkyl, halogen, C1-4 alkoxy or cyano;
a plurality of R¹⁵ may be the same or different;
a plurality of R²¹ may be the same or different;
each hydrogen atom may be a deuterium atom or a tritium atom;
with the proviso that ((1R, 5S, 6r)-6-(Cyclopropanecarbonyl)-3-azabicyclo [3.1.0]hexan-3-yl) (5-isopropyl-1H-pyrazol-3-yl) methanone, (5-Isopropyl-1H-pyrazol-3-yl)-[(1R, 5S)-6-[(2R)-2-methylpyrrolidine-1- carbonyl]-3-azabicyclo[3.1.0]hexan-3-yl] methanone, (5-Isopropyl-1H-pyrazol-3-yl)-[(1S, 5R) -6-[(2S)-2-methylpyrrolidine-1-carbonyl]-3-azabicyclo[3.1.0]hexan-3-yl] methanone, [(1S, 5R)-6-(2, 2-Dimethylpyrrolidine-1-carbonyl)-3-azabicyclo[3.1.0]hexan-3-yl]-(5-isopropyl-1H-pyrazol-3-yl) methanone and (5-Isopropyl-1H-pyrazol-3-yl)-[(1S, 5R)-6-(5-methyl-4-phenyl-isoxazol-3-yl)-3-azabicyclo [3.1.0]hexan-3-yl] methanone are excluded; or a salt thereof.

Other KDM5A inhibitors include NCDM-81a and NCDM-82a, CPI-455, 7-oxo-5-phenyl-6-propan-2-yl-1H-pyrazolo[1,5-a]pyrimidine-3-carbonitrile, KDM5-C49, GSK467, N71, compounds 10 and 13 (below), and other compounds as disclosed in Miyake et al, Bioorganic & Medicinal Chemistry 27 (February 2019) 1119-1129, which is incorporated by reference.

| | |
|---|---|
| | 10 |
| 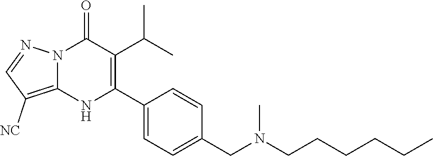 [SMILES: CCCCCCN(C)Cc1ccc(-c2[nH]c3c(C#N)cnn3c(=O)c2C(C)C)cc1] | |
| 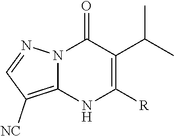 | |

| Entry | Compound | R | IC₅₀ (nM) |
|---|---|---|---|
| 1 | 1a | — | 2700 ± 210 |
| 2 | 3 | Ph | 506 ± 38 |
| 3 | 7 | — | 170 ± 12 |
| 4 | 10 | 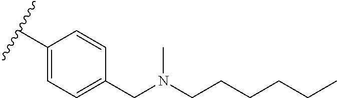 [SMILES: *c1ccc(CN(C)CCCCCC)cc1] | 22.7 ± 3.2 |
| 5 | 11 | 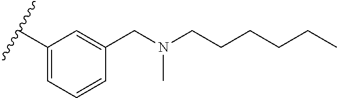 [SMILES: *c1cccc(CN(C)CCCCCC)c1] | 65.3 ± 8.6 |
| 6 | 12 | 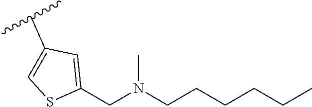 [SMILES: *c1csc(CN(C)CCCCCC)c1] | 179 ± 12 |
| 7 | 13 | 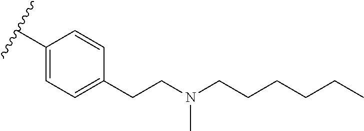 [SMILES: *c1ccc(CCN(C)CCCCCC)cc1] | 4.37 ± 0.19 |

Additional KDM5A inhibitors include ryuvidine (Mitsui et al, Sci Rep. 2019 Jul. 9;9(1):9952); the pyrazolylpyradines (including compound 33) disclosed in Nie et al, Bioorganic & Medicinal Chemistry Letters 28 (2018) 1490-1494, e.g., compounds having the structure

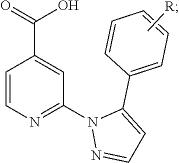

the ortho-substituted anilides disclosed in Jaikhan et al, Bioorganic & Medicinal Chemistry Letters 29 (March 2019) 1173-1176. In another embodiment, the KDM5A inhibitor is JQKD82. See, Ohguchi et al, Lysine Demethylase 5A Is Required for MYC-Driven Transcription in Multiple Myeloma, Blood Cancer Discov. 2021 July; 2(4): 370-387 (epub Apr. 10, 2021), which is incorporated herein by reference.

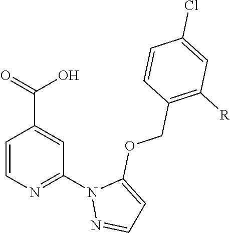

| | | | | | 2k-75-t [symbol] |
|---|---|---|---|---|---|
| | | KDM5A^{a} | KDM5B^{a} | KDM4C^{a} | H3K4me3 |
| Compd | k | IC₅₀ (μM) | IC₅₀ (μM) | IC₅₀ (μM) | EC₅₀ (μM) |
| 21 | B | 0.014 | 0.004 | 0.054 | 0.42 |
| 32 | F [symbol] | 0.018 | 0.004 | — | 1.3 |
| 33 | Me | 0.013 | 0.002 | 0.041 | 0.09 |
| 34 | OMe | 0.012 | 0.003 | 0.038 | 0.31 |
| 35 | OEt | 0.018 | 0.004 | 0.078 | 1.0 |
| 36 | OCH₂cPγ | 0.025 | 0.036 | 0.28 | — |
| 37 | OBn | 0.032 | 0.004 | 0.074 | 0.8 |

^{a}Values derived from biochemical assays with pre-incubation. [symbol]
indicates data missing or illegible when filed

Additional KDM5A inhibitors include those identified in Yang GJ, et al, Structure-Based Discovery of a Selective KDM5A Inhibitor that Exhibits Anti-Cancer Activity via Inducing Cell Cycle Arrest and Senescence in Breast Cancer Cell Lines. Cancers (Basel). 2019 Jan. 15;11(1):92. doi:10.3390/cancers11010092. PMID: 30650517; PMCID: PMC6360022. In one embodiment, the KDM5A inhibitor is

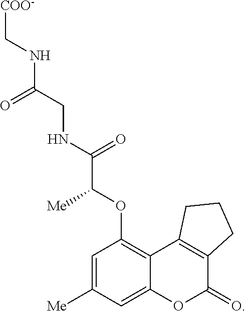

Additional KDM5A inhibitors include those identified in McAllister et al, Recent Progress in Histone Demethylase Inhibitors, J. Med. Chem. 2016, 59, 4, 1308-1329 (December 2015). In another embodiment, the KDM5A inhibitor is 5-Chloro-2-[(E)-2-[phenyl(pyridin-2-yl)methylidene]hydrazin-1-yl]pyridine (JIB04). See US 2020/0155526, which is incorporated herein by reference. Additional KDM5A inhibitors include those described in WO 2021/010492, which is incorporated herein by reference.

Additional KDM5A inhibitors include isonicotonic acid derivatives, pyrido-pyrimidine compounds, 3-thio-1,2, 4-triazole compounds, cyclopenta[c]chromen-based compounds, pyrazolo-pyrimidin compounds, 2,4-pyridinedicarboxylic acid analogs, pyrido-pyrimidinone compounds, cyclometalated rhodium(III) complexes and benzothiazoledione derivatives. In further embodiments, the inhibitor is selected from YUKA1, YUKA2, ZINC2140392, 8-(4-(2-(4-(3,5-dichlorophenyl)piperidin-1-yljcthyl)-1 H-pyrazol-1-yl)pyrido[3,4-d]pyrimidin-4(3H)-one, 8-(4-(2-(4-(3-chlorophenyl)piperidin-1-yljcthyl)-1H-pyrazol-1- yl)pyrido [3,4-d]pyrimidin-4(3H)-one, CPI-455, 5-[1-(1,1-dimethylethyl)-1H-pyrazol-4-yl]-4,7-dihydro-6-(1-methylethyl)-7-oxo-pyrazolo[1,5-a]pyrimidine-3-carbonitrile, KDMC-C49, KDMC-70, 2-[(1-Benzyl-1H-pyrazol-4-yl)oxy]-pyrido-[3,4-d]pyrimidin-4(3H)-one, KDOAM-25, N-(3-((methyl(2-(1-(4-oxo-3,4-dihydropyrido [3 ,4-d]pyrimidin-8-yl)-1H-pyrazol-4-yl)ethyl)amino) methyl)phenyl)acrylamide, cyclometalated rhodium (III) complex 1, and ryuvidine (see, WO 2021/116372, which is incorporated herein by reference). Additional examples of KDM5 inhibitors include the compounds disclosed in WO2016/057924 (Genentech/Constellation Pharmaceuticals), US20140275092 (Genentech/Constellation Pharmaceuticals), US2014/0371195 (Epitherapeutics), US2014/0371214 (Epitherapeutics), US2016/0102096 (Epitherapeutics), US2014/0194469 (Quanticel), US2014/0171432, US2014/0213591 (Quanticel), US2016/039808 (Quanticel), US2014/0275084 (Quanticel), and WO2014/164708 (Quanticel), and WO2020/028097 Gilead Sciences, each of which is incorporated herein by reference. In other embodiments, the KDM5A inhibitor has the following structure:

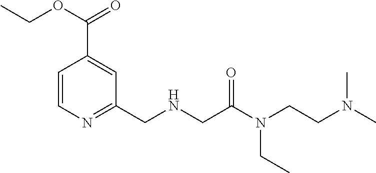 [SMILES: CCOC(=O)c1ccnc(CNCC(=O)N(CC)CCN(C)C)c1]

or a pharmaceutically acceptable salt thereof. See US 2018/0042905, which is incorporated herein by reference.

In other embodiments, the KDM5A inhibitor has the structure of formula (II) below:

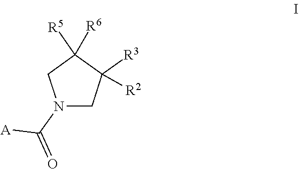

or a salt thereof, wherein:
A is selected from the group consisting of:

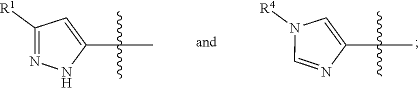

R¹ is halo, —N(R^{x})₂, C₁₋₆alkyl, C₂₋₆alkenyl, C₂₋₆alkynyl, 3-8 membered carbocyclyl, C₁₋₆alkoxy, 5-10 membered aryl, 5-10 membered heteroaryl, or 3-8 membered heterocyclyl, wherein said C₁₋₆alkyl, C₁₋₆alkenyl, C₁₋₆alkynyl, 3-8 membered carbocyclyl, C1-6alkoxy, 5-membered aryl, 5-10 membered heteroaryl, and 3-8 membered heterocyclyl are optionally substituted with one or more groups independently selected from the group consisting of oxo, hydroxy, halo, C₁₋₃alkoxy, 3-8 membered carbocyclyl, 5-10 membered aryl, —N(R^{x})₂, —N(R^{x})C(O)R^{x}, and C1-3alkyl;
each R^{x} is independently selected from the group consisting of H and C₁₋₆alkyl, that is optionally substituted with one or more groups independently selected from the group consisting of halo, hydroxy, and C1-3alkoxy;
R² is 5-10 membered carbocyclyl, 5-10 membered heterocyclyl, 5-10 membered aryl, 5-10 membered heteroaryl, —OR^{a}, —C(O)N(R^{a})₂, or NR^{a}R^{b}, wherein each 5-10 membered carbocyclyl, 5-10 membered heterocyclyl, 5-10 membered aryl, and 5-10 membered heteroaryl is optionally substituted with one or more groups R^{d};
R^{a} and R^{b} are each independently selected from the group consisting of H, C₁₋₆alkyl, C₂₋₆alkenyl, C₂₋₆alkynyl, 3-8 membered carbocyclyl, 3-8 membered heterocyclyl, 5-10 membered aryl, 5-10 membered heteroaryl —C(O)R^{c}, —CO₂R^{c}, —C(O)N(R^{c})₂, C(O)SR^{c}, and —C(O)C(O)R^{c}, wherein each C₁₋₆alkyl, C₂₋₆alkenyl, C₂₋₆alkynyl, 3-8 membered carbocyclyl, 3-8 membered heterocyclyl, 5-10 membered aryl, and 5-10 membered heteroaryl is optionally substituted with one or more groups independently selected from the group consisting of halo, nitro, cyano, oxo, C₁₋₄alkyl, C₂₋₄alkenyl, C₂₋₄alkynyl, 3-8 membered carbocyclyl, —OR^{c}—N(R^{c})₂, —C(O)R^{c}, —CO₂R^{c}, —C(O)N(R^{c})₂, —C(O)SR^{c}, —C(O)C(O)R^{c}, —S(O)R^{c}, —SO₂R^{c}, —SO₂N(R^{c})₂, —N(R^{c})C(O)R^{c}, —N(R^{c})C(O)N(R^{c})₂, —N(R^{c})SO₂R^{c}, —N(R^{c})SO₂N(R^{c})₂, —N(R^{c})N(R^{c})₂, —N(R^{c})C(═N(R^{c}))N(R^{c})₂, —C(═N)N(R^{c})₂, —C═NOR^{c}, and —C(═N(R^{c}))N(R^{c})₂;
each R^{c} is independently selected from the group consisting of H, C₁₋₆alkyl, C₂₋₆alkenyl, C₂₋₆alkynyl, 3-8 membered carbocyclyl, 3-8 membered heterocyclyl, 5-10 membered aryl and 5-10 membered heteroaryl, wherein each C₁₋₆alkyl, C₂₋₆alkenyl, C₂₋₆alkynyl, 3-8 membered carbocyclyl, 3-8 membered heterocyclyl, 5-10 membered aryl and 5-10 membered heteroaryl is optionally substituted with one or more groups R^{h};
each R^{d} is independently selected from the group consisting of halo, nitro, cyano, oxo, C₁₋₆alkyl, C₂₋₆alkenyl, C₂₋₆alkynyl, 3-8 membered carbocyclyl, 3-8 membered heterocyclyl, 5-10 membered aryl, 5-10 membered heteroaryl, —OR^{e}, —N(R^{e})₂, —C(O)R^{e}, —CO₂R^{e}, —C(O)N(Re)₂, —C(O)SR^{e}, —C(O)C(O)R^{e}, —S(O)R^{e}, —SO₂R^{e}, —SO₂N(R^{e})₂, —N(R^{e})C(O)R^{e}, —N(R^{e})C(O)N(R^{e})₂, —N(R^{e})SO₂R^{e}, —N(R^{e})SO₂N(R^{e})₂, —N(R^{e})N(R^{e})₂, —N(R^{e})C(═N(R^{e}))N(R^{e})₂, —C(═N)N(R^{e})₂, —C═NOR^{e}, and —C(═N(R^{e}))N(R^{e})₂, wherein each C₁₋₆alkyl, C₂₋₆alkenyl, C₂₋₆alkynyl, 3-8 membered carbocyclyl, 3-8 membered heterocyclyl, 5-10 membered aryl and 5-10 membered heteroaryl is optionally substituted with one or more groups independently selected from R^{f};
each R^{e} is independently selected from the group consisting of H, C₁₋₆alkyl, C₂₋₆alkenyl, C₂₋₆alkynyl, 3-8 membered carbocyclyl, 3-8 membered heterocyclyl, 5-10 membered aryl and 5-10 membered heteroaryl, wherein each C₁₋₆alkyl, C₂₋₆alkenyl, C₂₋₆alkynyl, 3-8 membered carbocyclyl, 3-8 membered heterocyclyl, 5-10 membered aryl and 5-10 membered heteroaryl is optionally substituted with one or more groups independently selected from the group consisting of C₁₋₄alkyl, C₂₋₄alkenyl, C₂₋₄alkynyl, 3-8 membered carbocyclyl, 3-8 membered heterocyclyl, 5-10 membered aryl and 5-10 membered heteroaryl;
each R^{f} is independently selected from the group consisting of halo, nitro, cyano, oxo, C₁₋₄alkyl, C₂₋₄alkenyl, C₂₋₄alkynyl, 3-8 membered carbocyclyl, —OR^{g}, —SR^{g}, —N(R^{g})₂, —C(O)R^{g}, —CO₂R^{g}, —C(O)N(R^{g})₂, —C(O)SR^{g}, —C(O)C(O)R^{g}, —S(O)R^{g}, —SO₂R^{g}, —SO₂N(R^{g})₂, —N(R^{g})C(O)R^{g}, —N(R^{g})C(O)N(R^{g})₂, —N(R^{g})SO₂R^{g}, —N(R^{g})SO₂N(R^{g})₂, —N(R^{g})N(R^{g})₂, —N(R^{g})C(═N(R^{g}))N(R^{g})₂, —C(═N)N(R^{g})₂, and —C═NOR⁸, —C(═N(R))N(R^{g})₂, wherein each C₁₋₄alkyl, C₂₋₄alkenyl, C₂₋₄alkynyl, and 3-8 membered carbocyclyl is optionally substituted with one or more groups independently selected from the group consisting of halo, cyano, oxo, 3-8 membered carbocyclyl, —OR^{g}, —N(R^{g})₂, —C(O)R^{g}, —CO₂R^{g}, —C(O)N(R^{g})₂, —SO₂R^{g}, —SO₂N(R^{g})₂, and —N(R^{g})C(O)R^{e};
each R^{g} is independently selected from the group consisting of H, C₁₋₆alkyl, C₂₋₆alkenyl, C₂₋₆alkynyl, 3-8 membered carbocyclyl, 3-8 membered heterocyclyl, 5-10 membered aryl and 5-10 membered heteroaryl, wherein each C₁₋₆alkyl, C₂₋₆alkenyl, C₂₋₆alkynyl, 3-8 membered carbocyclyl, 3-8 membered heterocyclyl, 5-10 membered aryl and 5-10 membered heteroaryl is optionally substituted with one or more groups independently selected from the group consisting of C₁₋₄alkyl, C₂₋₄alkenyl, C₂₋₄alkynyl, 3-8 membered carbocyclyl, 3-8 membered heterocyclyl, 5-10 membered aryl and 5-10 membered heteroaryl; and
each R^{h} is independently selected from the group consisting of halo, nitro, cyano, oxo, C₁₋₄alkyl, C₂₋₄alkenyl, C₂₋₄alkynyl, 3-8 membered carbocyclyl, 3-8 membered heterocyclyl, 5-10 membered aryl, 5-10 membered heteroaryl, —N(R^{k)}₂, and, —OR^{K}, wherein each C₁₋₄alkyl, C₂₋₄alkenyl, C₂₋₄alkynyl, 3-8 membered carbocyclyl, 3-8 membered heterocyclyl, 5-10 membered aryl and 5-10 membered heteroaryl is optionally substituted with one or more groups independently selected from the group consisting of halo, hydroxy, C₁₋₄alkoxy, cyano, oxo, C₁₋₄alkyl, C₂₋₄alkenyl, C₂₋₄alkynyl, 3-8 membered carbocyclyl, and 5-10 membered aryl; each Rkis independently selected from the group consisting of H, C₁₋₄alkyl, C₂₋₄alkenyl, C₂₋₄alkynyl, 3-8 membered carbocyclyl, and 5-10 membered aryl wherein any C₁₋₄alkyl, C₁₋₄alkenyl, C₂₋₄alkynyl, 3-8 membered carbocyclyl, and 5-10 membered aryl carbocyclyl is optionally substituted with one or more groups independently selected from the group consisting of halo, cyano, oxo hydroxy, and 3-8 membered carbocyclyl; and
R³ is H or C₁₋₆alkyl;
R⁴ is C₁₋₆alkyl, C₂₋₆alkenyl, C₂₋₆alkynyl, or 3-8 membered carbocyclyl, 5-10 membered aryl, 5-10 membered heteroaryl wherein each C₁₋₆alkyl, C₂₋₆alkenyl, C₂₋₆alkynyl, and 3-8 membered carbocyclyl, 5-10 membered aryl, 5-10 membered heteroaryl is optionally substituted with one or more groups independently selected from halo, hydroxy, cyano, C₁₋₆alkoxy, and 3-8 membered carbocyclyl; and
R⁵ is H, halo, or C₁₋₆alkyl, and R⁶ is H, C₁₋₆alkyl, C₂₋₆alkenyl, C₂₋₆alkynyl, halo, hydroxy, or 3-8 membered carbocyclyl, wherein each C₁₋₆alkyl, C₂₋₆alkenyl, C₂₋₆alkynyl, and 3-8 membered carbocyclyl, is optionally substituted with one or more groups independently selected from halo, hydroxy, cyano, C₁₋₆alkoxy, and 3-8 membered carbocyclyl; or R⁵ and R⁶ taken together with the atom to which they are attached form a 3-8 membered carbocyclyl or a 3-8 membered heterocyclyl, which 3-8 membered carbocyclyl and 3-8 membered heterocyclyl are optionally substitutes with one or more groups independently selected from halo, hydroxy, cyano, C₁₋₆alkoxy, and 3-8 membered carbocyclyl. See US 2017/0312252 which is incorporated herein by reference.

In other embodiments, the KDM5A inhibitor is N-(3-ethynylphenyl)-6,7-bis(2-methoxyethoxy)quinazolin-4-amine or a pharmaceutically acceptable salt thereof (e.g., erlotinib) or other compound described in US 2016/0143910, which is incorporated herein by reference.

In certain embodiments, the KDM5A inhibitor is a compound, or a salt or solvate, thereof, having the structure of (I), (II), (III), or (IV):

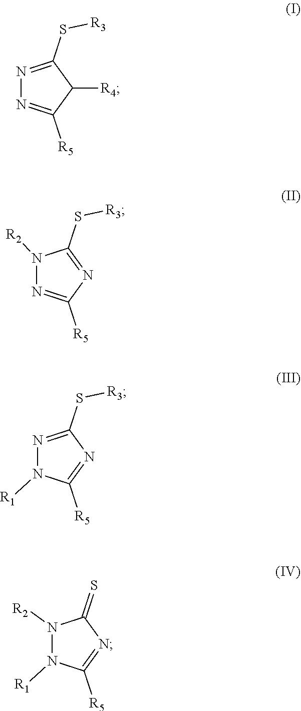

wherein in formulae (I)-(IV):
R1, R2, and R5 are each independently selected from the group consisting of H, C1-C6 alkyl, aryl-(Ci-C3)alkyl, substituted aryl-(Ci-C3)alkyl, substituted C1-C6 alkyl, Ci-C6 haloalkyl, C3-C7 cycloalkyl, substituted C3-C7 cycloalkyl, aryl, substituted aryl, heterocyclyl, substituted heterocyclyl, heteroaryl, and substituted heteroaryl;
R3 is selected from the group consisting of H, —C(0)R6, and —SR8;
R4 is selected from the group consisting of H, Ci-C6 alkyl, substituted Ci-C6 alkyl, d-Ce alkenyl, aryl-(Ci-C3)alkyl, substituted aryl-(Ci-C3)alkyl,
heteroaryl-(Ci-C3)alkyl, substituted heteroaryl-(Ci-C3)alkyl, Ci-C6 haloalkyl, C3-C7 cycloalkyl, substituted C3-C7 cycloalkyl, aryl, substituted aryl, heterocyclyl, substituted heterocyclyl, heteroaryl, substituted heteroaryl, and —NHR7;
R6 is selected from the group consisting of Ci-C6 alkyl, aryl, and heteroaryl; R7 is selected from the group consisting of H, Ci-C6 alkyl, substituted Ci-C6 alkyl, aryl, substituted aryl, aryl-(Ci-C3)alkyl, substituted aryl-(Ci-C3)alkyl, heteroaryl, substituted heteroaryl, heterocyclyl, substituted heterocyclyl, —C(0)R9, —S(0)R9, —S(0)2R9;
R8 is selected from the group consisting of H, Ci-C6 alkyl, substituted Ci-C6 alkyl, C2-C6 alkenyl, C3-C7 cycloalkyl, substituted C3-C7 cycloalkyl, aryl, substituted aryl, heterocyclyl, substituted heterocyclyl, heteroaryl, and substituted heteroaryl; and
R9 is selected from the group consisting of Ci-C6 alkyl, substituted Ci-C6 alkyl, C3-C7 cycloalkyl, substituted C3-C7 cycloalkyl, aryl, substituted aryl, heterocyclyl, substituted heterocyclyl, heteroaryl, and substituted heteroaryl. See WO 2017/197210 A1, which is incorporated herein by reference.

In certain embodiments, the KDM5A inhibitor is (R)-2-hydroxyglutarate. See Gunn K, et al. (R)-2-Hydroxyglutarate Inhibits KDM5 Histone Lysine Demethylases to Drive Transformation in IDH-Mutant Cancers. Cancer Discov. 2023 Jun. 2;13(6):1478-1497, which is incorporated herein by reference.

In certain embodiments, the KDM5A inhibitor is a compound that is an isonicotinic acid inhibitor or a prodrug thereof. In certain embodiments, the prodrug is GS-5801. See Gilmore SA, et al. Characterization of a KDM5 small molecule inhibitor with antiviral activity against hepatitis B virus. PLOS One. 2022 Dec 7;17(12):e0271145, which is incorporated herein by reference.

In certain embodiments, the KDM5A inhibitor is PBIT, KDM5-C49, KDM5-C70, GDK467, KDOAM-25, CPI-455, KDM5-Inh1, and KDM5-Inh1A. See Yoo J, et al. Drawing a line between histone demethylase KDM5A and KDM5B: their roles in development and tumorigenesis. Exp Mol Med. 2022 Dec;54(12):2107-2117, which is incorporated herein by reference.

Additional KDM5A inhibitors include those identified in Yang GJ, et al. The emerging role of KDM5A in human cancer. J Hematol Oncol. 2021 Feb. 17;14(1):30, which is incorporated herein by reference.

In certain embodiments, inhibiting or reducing KDM5A in the subject can be accomplished by reducing the amount of mRNA, e.g., via RNAi, or protein in the subject. Thus, in one embodiment, KDM5A is downregulated by reducing the level of KDM5A mRNA in the subject. KDM5A mRNA levels may be reduced, in one embodiment, using siRNA. siRNA can be generated against KDM5A using sequences known in the art. For example, the following KDM5A sequence can be found in GenBank: NP_001036068.1 which is incorporated herein by reference.

In certain embodiments, inhibiting or reducing KDM5A in the subject can be accomplished by reducing the amount of KDM5A protein in the subject via administration of an antibody that neutralizes or blocks the action of KDM5A (e.g., an anti-KDM5A antibody).

### Methods

Provided herein, in one embodiment is a method of increasing intratumor CD8⁺ T-cell infiltration in a subject in need thereof. The method includes administering a KDM5A inhibitor to a subject.

In another embodiment, a method of augmenting tumor antigen processing and/or presentation in a subject in need thereof is provided. The method includes administering a KDM5A inhibitor to a subject.

In another embodiment, a method of treating epithelial ovarian cancer in a subject in need thereof is provided. The method includes administering a KDM5A inhibitor and a cytotoxic agent to a subject.

In another embodiment, a method of increasing the number or activation of CD8⁺ T-cells in a subject in need thereof is provided. The method includes administering a KDM5A inhibitor to a subject.

In yet another embodiment, a method of treating cancer in a subject in need thereof is provided. The method includes administering a KDM5A inhibitor and interferon gamma to a subject.

In another embodiment, a method of increasing the expression of the MHC class I complex, or component thereof, in a subject in need thereof is provided. The method includes administering a KDM5A inhibitor to a subject.

In one embodiment, the effective amount of the KDM5A inhibitor is an amount ranging from about 0.01 mg to about 10 mg, including all amounts therebetween and end points. In one embodiment, the effective amount of the KDM5A inhibitor is about 0.1 mg/kg to about 5 mg/kg, including all amounts therebetween and end points. In another embodiment, the effective amount of the KDM5A inhibitor is about 0.3 mg/kg to about 1.0 mg/ml, including all amounts therebetween and end points. In another embodiment, the effective amount of the KDM5A inhibitor is about 0.3 mg/ kg. In another embodiment, the effective amount of the KDM5A inhibitor is about 0.4 mg/ kg. In another embodiment, the effective amount of the KDM5A inhibitor is about 0.5 mg/ kg. In another embodiment, the effective amount of the KDM5A inhibitor is about 0.6 mg/ kg. In another embodiment, the effective amount of the KDM5A inhibitor is about 0.7 mg/ kg. In another embodiment, the effective amount of the KDM5A inhibitor is about 0.8 mg/ kg. In another embodiment, the effective amount of the KDM5A inhibitor is about 0.9 mg/ kg. In another embodiment, the effective amount of the KDM5A inhibitor is about 1.0 mg/ kg.

In one embodiment, the effective amount of the KDM5A inhibitor is about 0.1 mg to about 5 mg, including all amounts therebetween and end points. In another embodiment, the effective amount of the KDM5A inhibitor is about 0.3 mg to about 1.0 mg, including all amounts therebetween and end points. In another embodiment, the effective amount of the KDM5A inhibitor is about 0.3 mg. In another embodiment, the effective amount of the KDM5A inhibitor is about 0.4 mg. In another embodiment, the effective amount of the KDM5A inhibitor is about 0.5 mg. In another embodiment, the effective amount of the KDM5A inhibitor is about 0.6 mg. In another embodiment, the effective amount of the KDM5A inhibitor is about 0.7 mg. In another embodiment, the effective amount of the KDM5A inhibitor is about 0.8 mg. In another embodiment, the effective amount of the KDM5A inhibitor is about 0.9 mg. In another embodiment, the effective amount of the KDM5A inhibitor is about 1.0 mg.

In one embodiment, the effective amount of the KDM5A inhibitor is an amount ranging from about 1 μM to about 2mM, including all amounts therebetween and end points. In one embodiment, the effective amount of the KDM5A inhibitor is about 10 μM to about 100 μM, including all amounts therebetween and end points. In another embodiment, the effective amount of the KDM5A inhibitor is about 5 μM. In another embodiment, the effective amount of the KDM5A inhibitor is about 10 μM. In another embodiment, the effective amount of the KDM5A inhibitor is about 20 μM. In another embodiment, the effective amount of the KDM5A inhibitor is about 50 μM. In another embodiment, the effective amount of the KDM5A inhibitor is about 100 μM. In another embodiment, the effective amount of the KDM5A inhibitor is about 200 μM. In another embodiment, the effective amount of the KDM5A inhibitor is about 300 μM. In another embodiment, the effective amount of the KDM5A inhibitor is about 400 μM. In another embodiment, the effective amount of the KDM5A inhibitor is about 500 μM. In another embodiment, the effective amount of the KDM5A inhibitor is about 600 μM. In another embodiment, the effective amount of the KDM5A inhibitor is about 700 μM. In another embodiment, the effective amount of the KDM5A inhibitor is about 800 μM. In another embodiment, the effective amount of the KDM5A inhibitor is about 900 μM. In another embodiment, the effective amount of the KDM5A inhibitor is about 1 mM. In another embodiment, the effective amount of the KDM5A inhibitor is about 1.25 mM. In another embodiment, the effective amount of the KDM5A inhibitor is about 1.5 mM. In another embodiment, the effective amount of the KDM5A inhibitor is about 1.75 mM. In another embodiment, the effective amount of the KDM5A inhibitor is about 2 mM.

In certain embodiments, the cancer treated includes, but is not limited to, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma or Merkel cell carcinoma), and pancreas, as well as adenocarcinomas which include malignancies such as colon cancers, rectal cancer, renal-cell carcinoma, liver cancer, non-small cell lung cancer, cancer of the small intestine, cancer of the esophagus. The cancer may be at an early, intermediate, late stage or metastatic cancer. In certain embodiments, the cancer is ovarian cancer. In certain embodiments, the cancer is high grade serous ovarian cancer (HGSOC).

In one embodiment, the cancer is chosen from a lung cancer (e.g., a non-small cell lung cancer (NSCLC) (e.g., a NSCLC with squamous and/or non-squamous histology, or a NSCLC adenocarcinoma)), a skin cancer (e.g., a Merkel cell carcinoma or a melanoma (e.g., an advanced melanoma)), a kidney cancer (e.g., a renal cancer (e.g., a renal cell carcinoma (RCC) such as a metastatic RCC or clear cell renal cell carcinoma (CCRCC)), a liver cancer, a myeloma (e.g., a multiple myeloma), a prostate cancer (including advanced prostate cancer), a breast cancer (e.g., a breast cancer that does not express one, two or all of estrogen receptor, progesterone receptor, or Her2/neu, e.g., a triple negative breast cancer), a colorectal cancer, a pancreatic cancer, a head and neck cancer (e.g., head and neck squamous cell carcinoma (HNSCC), a brain cancer (e.g., a glioblastoma), an endometrial cancer, an anal cancer, a gastro-esophageal cancer, a thyroid cancer (e.g., anaplastic thyroid carcinoma), a cervical cancer, a neuroendocrine tumor (NET) (e.g., an atypical pulmonary carcinoid tumor), a lymphoproliferative disease (e.g., a post-transplant lymphoproliferative disease) or a hematological cancer, T-cell lymphoma, B-cell lymphoma, a non-Hodgkin lymphoma, or a leukemia (e.g., a myeloid leukemia or a lymphoid leukemia). In yet another embodiment, the cancer is a hepatocarcinoma, e.g., an advanced hepatocarcinoma, with or without a viral infection, e.g., a chronic viral hepatitis. In a certain embodiment, the subject has been treated previously for cancer.

In certain embodiments, the methods described herein include treatment in combination with another cancer treatment or therapeutic agent, including known chemotherapeutic agents. As used herein, the term chemotherapeutic agent is used interchangeably with cytotoxic agent.

In certain embodiments, the KDM5A inhibitor is provided in conjunction with a checkpoint inhibitor. Immune checkpoints represent significant barriers to activation of functional cellular immunity in cancer, and antagonistic antibodies specific for inhibitory ligands on T cells including CTLA4 and programmed death-1 (PD-1) are examples of targeted agents being evaluated in the clinics. In one embodiment, the subject has previously received checkpoint therapy, prior to receiving KDM5A inhibitor therapy. The subject may, in some embodiments, receive the same or different checkpoint therapy after administration of the KDM5A inhibitor.

Immune checkpoint molecules that may be targeted for blocking or inhibition include, but are not limited to, CTLA-4, 4-1BB (CD137), 4-1BBL (CD137L), PDL1, PDL2, PD1, CD134, B7-H3, B7-H4, BTLA, HVEM, TIM3, GAL9, LAG3, TIM3, B7H3, B7H4, VISTA, KIR, 2B4, CD160 (also referred to as BY55) and CGEN-15049. In one embodiment, the checkpoint inhibitor is PD-1 inhibitor. In another embodiment, the checkpoint inhibitor is PD-L1 inhibitor.

Immune checkpoint inhibitors include antibodies, or antigen binding fragments thereof, or other binding proteins, that bind to and block or inhibit the activity of one or more of CTLA-4, PDL1, PDL2, PD1, CD134, B7-H3, B7-H4, BTLA, HVEM, TIM3, GAL9, LAG3, TIM3, B7H3, B7H4, VISTA, KIR, 2B4, CD160 and CGEN-15049. Suitable immune checkpoint inhibitors include those that block PD-1, such as pembrolizumab, nivolumab, AGEN 2034, BGB-A317, BI-754091, CBT-501 (genolimzumab), MEDI0680, MGA012, PDR001, PF-06801591, REGN2810 (SAR439684), and TSR-042. MK-3475 (PD-1 blocker) Nivolumab, CT-011.

Immune checkpoint inhibitors also include those that block PD-L1, such as durvalumab, atezolizumab, avelumab, and CX-072. Other suitable inhibitors include Anti-B7-H1 (MEDI4736), AMP224, BMS-936559, MPLDL3280A, and MSB0010718C.

In certain embodiment, the checkpoint inhibitor blocks CTLA-4. In certain embodiments, the checkpoint inhibitor is AGEN 1884, ipilimumab, or tremelimumab.

In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAGS antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-LAG-3 antibody selected from the group consisting of: BMS-986016 and LAG525. In some embodiments, the additional therapeutic agent is an anti-OX-40 antibody selected from: MEDI6469, MEDI0562, and MOXR0916. In some embodiments, the additional therapeutic agent is the anti-4-1BB antibody PF-05082566.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example, it is known that lambrolizumab is also known under the alternative and equivalent names MK-3475 and pembrolizumab.

In addition, more than one immune checkpoint inhibitor (e.g., anti-PD-1 antibody and anti-CTLA-4 antibody) may be used in combination with a KDM5A inhibitor.

The subject is treated with an effective amount of the checkpoint inhibitor. It should be understood that the “effective amount” for the checkpoint inhibitor may vary depending upon the agent(s) selected for use in the method, and may be determined by the person of skill in the art. In one embodiment an effective amount for the checkpoint inhibitor includes without limitation about lug to about 25 mg. In one embodiment, the range of effective amount is 0.001 to 0.01 mg. In another embodiment, the range of effective amount is 0.001 to 0.1 mg. In another embodiment, the range of effective amount is 0.001 to 1 mg. In another embodiment, the range of effective amount is 0.001 to 10 mg. In another embodiment, the range of effective amount is 0.001 to 20 mg. In another embodiment, the range of effective amount is 0.01 to 25 mg. In another embodiment, the range of effective amount is 0.01 to 0.1 mg. In another embodiment, the range of effective amount is 0.01 to 1 mg. In another embodiment, the range of effective amount is 0.01 to 10 mg. In another embodiment, the range of effective amount is 0.01 to 20 mg. In another embodiment, the range of effective amount is 0.1 to 25 mg. In another embodiment, the range of effective amount is 0.1 to 1 mg. In another embodiment, the range of effective amount is 0.1 to 10 mg. In another embodiment, the range of effective amount is 0.1 to 20 mg. In another embodiment, the range of effective amount is 1 to 25 mg. In another embodiment, the range of effective amount is 1 to 5 mg. In another embodiment, the range of effective amount is 1 to 10 mg. In another embodiment, the range of effective amount is 1 to 20 mg. Still other doses falling within these ranges are expected to be useful. The effective amount of the checkpoint inhibitor may be individually chosen based on the agent selected and other factors, e.g., size of the patient, type of cancer, etc.

In one embodiment, the KDM5A inhibitor and checkpoint inhibitor are administered approximately simultaneously. In another embodiment, the KDM5A inhibitor is administered prior to checkpoint inhibitor. In another embodiment, the KDM5A inhibitor is administered subsequent to the checkpoint inhibitor.

Chemotherapeutic agents (e.g., anti-cancer agents) are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol) and docetaxel; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CCN-U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; FUdR), and cytarabine (cytosine arabinoside), and purine analogues and related inhibitors such as mercaptopurine (6-mercaptopurine; 6-MP), thioguanine (6-thioguanine; 6-TG), and pentostatin (2′-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,p′-DDD) and aminoglutethimide; analogs thereof derivatives thereof and combinations thereof.

In one embodiment, the effective amount of the cytotoxic agent is an amount ranging from about 0.01 mg to about 10 mg, including all amounts therebetween and end points. In one embodiment, the effective amount of the cytotoxic agent is about 0.1 mg/kg to about 5 mg/kg, including all amounts therebetween and end points. In another embodiment, the effective amount of the cytotoxic agent is about 0.3 mg/kg to about 1.0 mg/ml, including all amounts therebetween and end points. In another embodiment, the effective amount of the cytotoxic agent is about 0.3 mg/ kg. In another embodiment, the effective amount of the cytotoxic agent is about 0.4 mg/ kg. In another embodiment, the effective amount of the cytotoxic agent is about 0.5 mg/ kg. In another embodiment, the effective amount of the cytotoxic agent is about 0.6 mg/ kg. In another embodiment, the effective amount of the cytotoxic agent is about 0.7 mg/ kg. In another embodiment, the effective amount of the cytotoxic agent is about 0.8 mg/ kg. In another embodiment, the effective amount of the cytotoxic agent is about 0.9 mg/ kg. In another embodiment, the effective amount of the cytotoxic agent is about 1.0 mg/ kg.

In one embodiment, the effective amount of the cytotoxic agent is about 0.1 mg to about 5 mg, including all amounts therebetween and end points. In another embodiment, the effective amount of the cytotoxic agent is about 0.3 mg to about 1.0 mg, including all amounts therebetween and end points. In another embodiment, the effective amount of the cytotoxic agent is about 0.3 mg. In another embodiment, the effective amount of the cytotoxic agent is about 0.4 mg. In another embodiment, the effective amount of the cytotoxic agent is about 0.5 mg. In another embodiment, the effective amount of the cytotoxic agent is about 0.6 mg. In another embodiment, the effective amount of the cytotoxic agent is about 0.7 mg. In another embodiment, the effective amount of the cytotoxic agent is about 0.8 mg. In another embodiment, the effective amount of the cytotoxic agent is about 0.9 mg. In another embodiment, the effective amount of the cytotoxic agent is about 1.0 mg.

In one embodiment, the effective amount of the cytotoxic agent is an amount ranging from about 1 μM to about 2mM, including all amounts therebetween and end points. In one embodiment, the effective amount of the cytotoxic agent is about 10 μM to about 100 μM, including all amounts therebetween and end points. In another embodiment, the effective amount of the cytotoxic agent is about 5μM. In another embodiment, the effective amount of the cytotoxic agent is about 10 μM. In another embodiment, the effective amount of the cytotoxic agent is about 20 μM. In another embodiment, the effective amount of the cytotoxic agent is about 50 μM. In another embodiment, the effective amount of the cytotoxic agent is about 100 μM. In another embodiment, the effective amount of the cytotoxic agent is about 200 μM. In another embodiment, the effective amount of the cytotoxic agent is about 300 μM. In another embodiment, the effective amount of the cytotoxic agent is about 400 μM. In another embodiment, the effective amount of the cytotoxic agent is about 500 μM. In another embodiment, the effective amount of the cytotoxic agent is about 600 μM. In another embodiment, the effective amount of the cytotoxic agent is about 700 μM. In another embodiment, the effective amount of the cytotoxic agent is about 800 μM. In another embodiment, the effective amount of the cytotoxic agent is about 900 μM. In another embodiment, the effective amount of the cytotoxic agent is about 1mM. In another embodiment, the effective amount of the cytotoxic agent is about 1.25 mM. In another embodiment, the effective amount of the cytotoxic agent is about 1.5 mM. In another embodiment, the effective amount of the cytotoxic agent is about 1.75 mM. In another embodiment, the effective amount of the cytotoxic agent is about 2 mM.

In certain embodiments, the KDM5A inhibitor is administered with interferon gamma (IFNγ).

In some embodiments, the level of KDM5A is detected in a sample obtained from a subject. This level may be compared to the level of a control. In one embodiment, an increase in the level of KDM5A as compared to a control indicates a greater risk of a poor prognosis in the subject. A poor prognosis, in some embodiments, may refer to a shorter life expectancy, higher tumor burden, or decreased likelihood of remission, as compared to an average subject with the same disease.

In one embodiment, when diagnosed with a poor prognosis, the subject is then treated for cancer. In one embodiment, the treatment includes an inhibitor of KDM5A. In another embodiment, the treatment includes a cytotoxic agent. In yet another embodiment, the treatment includes IFNγ.

Still further embodiments follow as “A” through “S”:
A. A method of increasing intratumor CD8⁺ T-cell infiltration in a subject in need thereof, the method comprising administering a KDM5A inhibitor to the subject.
B. A method of augmenting tumor antigen processing and/or presentation in a subject in need thereof, the method comprising administering a KDM5A inhibitor to the subject.
C. A method of treating epithelial ovarian cancer in a subject in need thereof, the method comprising administering a KDM5A inhibitor and a cytotoxic agent to the subject.
D. A method of increasing the number or activation of CD8⁺ T cells in a subject in need thereof, the method comprising administering a KDM5A inhibitor to the subject.
E. A method of treating cancer in a subject in need thereof, the method comprising administering a KDM5A inhibitor and interferon gamma to the subject.
F A method of treating cancer in a subject in need thereof, the method comprising administering a KDM5A inhibitor and a checkpoint inhibitor to the subject.
G. A method of increasing the expression of the MHC class I complex, or component thereof, in a subject in need thereof, the method comprising administering a KDM5A inhibitor to the subject.
H. The embodiment according to embodiment F, wherein the subject has a viral or bacterial infection.
I. The method according to any one of embodiments A to G, wherein the subject has ovarian cancer.
J. The method according to embodiment I, wherein the subject has high-grade serous ovarian cancer (HGSOC).
K. The method according to any one of embodiments A to G, wherein the subject has triple negative breast cancer.
L. The method according to any one of embodiments A to K, wherein the KDM5A inhibitor is CPI-455.
M. The method according to any one of embodiments A to L, wherein the KDM5A inhibitor is administered in an amount ranging from about 0.01 mg to 10 mg.
N. The method according to any one of embodiments A to K, comprising reducing the level of KDM5A mRNA in the subject.
O. The method according to embodiment N, wherein the KDM5A mRNA levels are reduced using siRNA.
P. A method of predicting poor prognosis in a subject having high grade serous ovarian cancer, the method comprising measuring the level of KDM5A in a sample from the subject, wherein an increased level of KDM5A as compared to a control is indicative of a poor prognosis.
Q. The method according to embodiment P, further comprising treating the subject.
R. The method according to embodiment Q, wherein the treatment comprises administration of a KDM5A inhibitor.
S. The method according to any one of embodiments P to R, wherein the sample is serum, plasma, or whole blood.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### EXAMPLES

### Example 1: Materials and Methods

Cell Culture, transfection, and reagents ID8 (RRID: CVCL_VA22), and HEK293FT (RRID: CVCL-0045) cells were cultured in DMEM supplemented with 10% fetal bovine serum (FBS) and 1% penicillin/streptomycin at 37° C. with 5% CO2. HGS2(RRID: CVCL_B5GW) (gift from Dr. Ronny Drapkin, University of Pennsylvania, Philadelphia, PA) were cultured in DMEM-F12 supplemented with 5% feral bovine serum (FBS),

Insulin/Transferrin/Selenium (Invitrogen, Cat #51300), 0.5 mg/ml Hydrocortisone (Sigma, Cat #H0135), 10 ng/ml Murine epidermal growth factor (Sigma, Cat #E4127) and 1% penicillin/streptomycin at 37° C.with 5% CO2. The human high-grade serous ovarian cancer (HGSOC) cell lines A1847 (RRID:CVCL_9724), PEO4 (RRID:CVCL_2690), OVSAHO (RRID:CVCL_3114), OVCAR10 (RRID:CVCL_4377), OVCAR3 (RRID:CVCL_0465), CAOV3 (RRID:CVCL_0201), COV382 (RRID:CVCL_2420), Kuramochi (RRID:CVCL_1345) were cultured in RPMI1640 (Corning, Cat #10-040-CM) supplemented with 10% FBS and 1% penicillin/streptomycin at 37° C. with 5% CO2. The human fallopian tube epithelial cells FT246 (RRID:CVCL UH61) and FT237 (RRID:CVCL_UH59) were gifts from Dr. Ronny Drapkin at the University of Pennsylvania (Philadelphia, PA), and they were grown in DMEM/F12 (Corning, Cat #10-092-CM) with 10% FBS. All cell lines were authenticated at The Wistar Institute Genomics Facility using short tandem repeat DNA profiling. Mycoplasma testing was performed using LookOut Mycoplasma PCR detection (Sigma, Cat# MP0035) every month. Cells were cultured for a maxixum of two months or 20 passages. Transfection was performed using Lipofectamine 2000 (Life Technologies, Cat #11668027) following the manufacturer's instructions. Doxycycline hyclate was purchased from Sigma (Cat #324385) and CPI-455 was purchased from Medkoo Biosiences (Cat #406987). Mouse recombinant interferon gamma (IFNγ) IFNγ was purchased from Stem Cell (Cat #78021.1). For in vivo bioluminescence imaging, DLuciferin potassium salt was purchased from Perkin Elmer (Cat #122799).

### Antibodies

For Western blotting, the following primary antibodies were used: mouse anti-β-actin (Sigma, Cat #A5316), rabbit anti-KDM5A (Abcam, Cat #ab194286, RRID: AB_1139986; CST, Cat #3876S, RRID: AB_2129055), mouse anti-H3K4me3 (Abcam, Cat #ab12209, RRID: AB_442957), anti-mouse IgG HRP (CST, Cat #7076, RRID: AB_330924), anti-rabbit IgG HRP (CST, Cat #7074, RRID: AB_2099233). For IHC, rabbit anti-KDM5A (CST, Cat#3876S, RRID: AB_2129055) was used. For in vivo mouse experiment, anti-mouse CD8 (BioXcell, Cat #BE0061, RRID: AB_10950145) and rat IgG2b isotype control (BioXcell, Cat #BE0090, RRID: AB_1107780) were used.

### CRISPR-Mediated Knockouts

pLentiCRISPR v2 (Addgene, Cat #52961, RRID: Addgene_52961) was digested with BsmBI (NEB, Cat #R0580) at 55° C. for 1 h and run on a 1% agarose gel. The digested plasmid was cut out and purified using QIAquick gel extraction kit (Qiagen, Cat #166047244). Each pair of oligos were phosphorylated using T4 PNK (NEB, Cat #M0201S) in T4 ligation buffer (New England Biolabs) and annealed in a thermocycler at 37° C. for 30 min, 95° C. for 5 min, ramped down to 25° C. at 5° C. per min. Annealed oligonucleotides were diluted 1:200 in RNase/DNase-free water. Ligation of the annealed oligonucleotide and digested pLentiCRISPR v2 plasmid was performed using Quick Ligase (NEB, Cat #M2200L).

Tet-Inducible shKdm5A Knockdown

tet-pLKO-shKdm5a was constructed by inserting the (the sense sequence is 5′-CCTTTGAGTGACTTAGAGGAA-3′ (SEQ ID NO: 1) for clone 1 or 5′-CAAAGAGAACAAGACGAGTTA-3′ (SEQ ID NO: 2) for clone 2) into the tet-pLKO-puro vector (Addgene, Cat #21915) digested with AgeI (NEB, Cat #R3552) and EcoRI restriction enzymes (NEB, Cat #R3010) and dephosphorylated for 30 min at 37° C. The digested plasmid was run on a 1% agarose gel, cut out, and purified using the Wizard SV Gel and PCR Clean Up kit (Promega, Cat #A9281). The oligonucleotides were phosphorylated using T4 PNK (NEB, Cat #M0201S) with T4 Ligation Buffer (NEB, Cat #B0202S). Samples were annealed in a thermocycler at 37° C.for 30 min and then at 95° C. for 5 min and finally were ramped down to 25° C. at 5° C. per min. Annealed oligonucleotides were diluted 1:200 in RNase/DNase-free water. Ligation of the annealed oligonucleotide and digested tet-pLKO-puro plasmid was performed using Quick Ligase (NEB, Cat #M2200S). To induce Kdm5a knockdown, cells infected with tet-inducible shKdm5a virus were treated with 2 μg/ml of Doxycycline (Sigma).

### Lentivirus Infection

HEK293FT cells were transfected with target vector, psPAX2 (Addgene, Cat #12260, RRID: Addgene_12260) and pCMV-VSV (Addgene, Cat #8454, RRID: Addgene_8454) by Lipofectamine 2000 for 6 h and replaced with fresh medium. Lentivirus was harvested and filtered with 0.45 μm filter 72 h post transfection. Cells were infected with lentiviral particles for 48 h and selected in medium containing 1 μg/ml puromycin.

### Immunoblots

Cells were trypsinized and washed two times with PBS. Protein was extracted with RIPA lysis buffer [50 mM Tris (pH 8.0), 150 mM NaCl, 1% Triton X-100, 0.5% sodium deoxycholate, 0.1% SDS and 1 mM phenylmethylsulfonyl fluoride (PMSF)] on ice for 30 min. Protein concentration was measured by the BCA assay (Pierce, Cat #23225). Samples were separated by SDS polyacrylamide gel electrophoresis (SDS-PAGE) and transferred to polyvinylidene fluoride membrane (Millipore, Cat #IPVH00010). Membranes were blocked with 4% BSA/TBS-T and then incubated with primary antibodies and secondary antibodies.

### Reverse-Transcriptase Quantitative PCR (RT-qPCR)

Total RNA was extracted using RNeasy Kit (Qiagen) following the manufacturer's instructions. Purified RNA was used for reverse-transcriptase PCR (RT-PCR) with High-Capacity cDNA Reverse Transcription Kit (Thermo Fisher, Cat #4374967). Quantitative PCR (qPCR) was performed using iTaq Universal SYBR Green Supermix (Bio-Rad, Cat #1725121) and run on QuantStudio 5 Real-Time PCR System.

### Chromatin Immunoprecipitation (ChIP)

ChIP was performed as previously described (28). The following antibodies were used for ChIP: rabbit anti-KDM5A (Abcam, Cat #ab 194286, RRID: AB_1139986, 5 ug per IP), mouse antiH3K4me3(Abcam, Cat #ab12209, RRID: AB_442957, 2 μg per IP). Isotype-matched IgGs were used as negative controls. ChIP DNA was purified by Zymo ChIP DNA clean and concentrator kit (Zymo research, Cat #D5205) and analyzed by qPCR.

### Flow Cytometry Analysis

For cell surface MHC class I H2-K1 expression analysis, ID8 or HGS2 cells were treated by 10mM EDTA (in DPBS) and washed twice with DPBS. Cells were then blocked by Fc blocking buffer (BD, Cat #553142, RRID: AB_394657) on ice for 30 min and then stained with PE-antiMHC-1 antibody (Abcam, Cat #ab25547, RRID: AB_470631) for another 30 min. After washing with DPBS twice, MHC-1 staining was detected by the Becton-Dickinson LSR18/LSR14 machine, and analyzed with FlowJo version 10 software (Tree Star, Inc.) For immune infiltration analysis, cells from ascites were collected, after lysis of red blood cells with RBC buffer (Biolegend, Cat #420302). The cells were sequentially filtered through 40 μm cell strainer. Tumor infiltrated lymphocytes (TILs) analysis were followed by viability staining (Thermo Fisher, Cat #L34957), Fc blocking (BD, Cat #553142, RRID:AB_394657) and then surface staining in FACS buffer (3% FBS in PBS) with fluorochrome-conjugated antibodies against: mouse CD45 (Biolegend, Cat#103147,RRID:AB_2564383), mouse CD3 (BD, Cat# 552774, RRID:AB_394460), mouse CD4 (Biolegend, Cat #100516, RRID:AB_312719), mouse CD8 (Biolegend, Cat #100708,

RRID: AB_312747), mouse CD19 (Biolegend, Cat #115523, RRID:AB_439718), mouse CD69 (Biolegend, Cat #104510, RRID:AB_313113), mouse PDL1(BioLegend, Cat #124321, RRID:AB_2563635), mouse PD-1 (Biolegend, Cat #135214, RRID:AB_10680238), mouse CD11c (Biolegend, Cat #117324, RRID:AB_830649), mouse CD11b (Biolegend, Cat #101259, RRID:AB_2566568). All FACS analyses were performed on a BD LSR II or a Canto II Flow Cytometer, and data were analyzed with FlowJo software (Tree Star, Inc., version 10).

### Immunohistochemistry (IHC)

HGSOC tumor tissue microarrays (TMA) were kindly provided by Dr. Benjamin G Bitler from The University of Colorado (COMIRB #17-7788). Detailed information on the tumors contained on the TMA has been published previously (29,30). IHC was performed using Dako En Vision+ system (Dako, Cat #K4002) following the manufacturer's instructions. Briefly, antigen retrieval was performed in sodium citrate buffer (Thermo Fisher, Car #005000) and boiled for 45 min. The sections were deparaffinized, rehydrated and immersed in 3% hydrogen peroxide in methanol to quench endogenous peroxidase activity. The sections were incubated with blocking buffer for 1 h, primary antibody against KDM5A (CST, #3876S, RRID: AB_2129055) at 4° C. overnight and secondary antibody for 1 h. Counterstaining was performed using Mayer's Hematoxylin (Dako, Cat #3309S).

### Syngeneic Ovarian Cancer Mouse Model

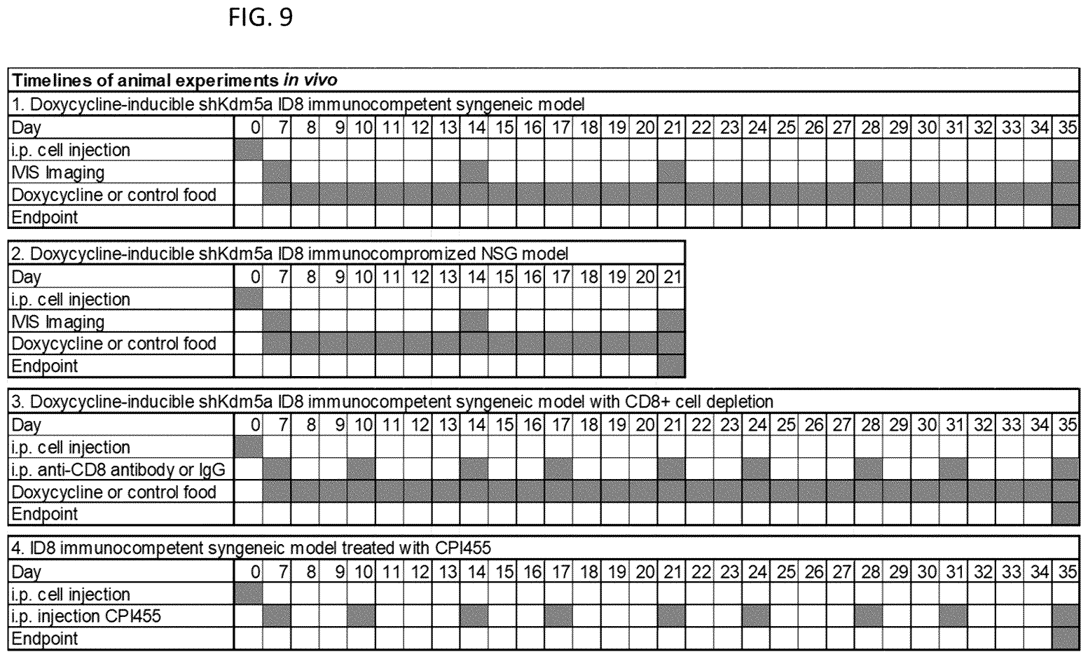

All animal protocols described in this study were approved by the Institutional Animal Care and Use Committee (IACUC) at The Wistar Institute. 6- to 8-week-old female wild-type C57BL/6 mice were purchased from Charles River Laboratories. ID8 cells were provided by K. Roby (Department of Anatomy and Cell Biology, University of Kansas) and retrovirally transduced to express luciferase. Briefly, 2×10⁶ of 70% confluent doxycycline-inducible shKdm5a ID8 cells were injected into the peritoneal cavity of mice and allowed to establish tumors. After 1 week, mice were randomized into two groups and treated with control (Bio-Serv, Cat #S4207) or doxycycline containing diet (Bio-Serv, #S3888) for 4 weeks. Tumor growth was followed by noninvasive imaging using an IVIS Spectrum. Images were analyzed using Live Imaging 4.0 software. After 4 weeks of treatment ascites volume was measured as an additional surrogate for tumor burden. For anti-CD8 antibody treatment, 2×10⁶ of 70% confluent doxycyclineinducible shKdm5a ID8 cells were injected into the peritoneal cavity of mice and allowed to establish tumors. After 1 week, mice were randomized into four groups and treated with control (Bio-Serv, Cat #S4207) or doxycycline containing diet (Bio-Serv, #S3888) in the presence of an anti-CD8 antibody (BioXcell, Cat#BE0061, RRID: AB_10950145) or a rat IgG2b isotype control (BioXcell, Cat #BE0090, RRID: AB_1107780), 500 μg per mouse, twice a week. For CPI-455 treatment, 2×10⁶ of 70% confluent ID8 cells were injected into the peritoneal cavity of mice and allowed to establish tumors. After 1 week, mice were randomized into two groups and treated with vehicle control or CPI-455 (Medkoo, Cat #406987) 50 mg/kg, twice a week. After 4 weeks of treatment ascites volume was measured as a surrogate for tumor burden. Timelines for all in vivo experiments are described in FIG. 9.

### CIBERSORT

CIBERSORT was used for the estimation of immune cell infiltration using TCGA HGSOC dataset (31). Gene expression profiles of the 300 cases was uploaded to CIBERSORT as a mixture file, and CIBERSORT was run with absolute mode option using LM22 signature gene file, 1000 permutations, and quantile normalization disabled. Pearson's correlation was used to test association between CD8+ T cell signature score and every gene expression in 300 TCGA HGSOC samples.

### RNA Sequencing

Total RNA of control ID8 and Kdm5a knockout ID8 cell lines was extracted using RNeasy mini Kit (Qiagen, Cat # 74106) and digested with DNase I (Qiagen, Cat #79254). RNA-seq libraries were constructed using ScriptSeq complete Gold kit (Epicentre, Cat #SCL24EP) and subjected to a 75 bp paired-end sequencing run on NextSeq 500, using Illumina's NextSeq 500 high output sequencing kit following the manufacturer's instructions.

### CUT&RUN Sequencing

For CUT&RUN sequencing, cells were harvested by trypsinization and gently washed twice using wash buffer (20 mM HEPES pH 7.5, 150 mM NaCl, 0.5 mM spermidine, and EDTA-free Protease Inhibitor Cocktail). Cells were then incubated with the antibody at 4° C. overnight in antibody buffer (wash buffer supplemented with 0.05% digitonin and 2 mM EDTA). The next day, supernatant was removed by centrifugation and cell pellets were washed once with Digwash buffer (Wash buffer containing 0.05% digitonin). Cell pellets were then incubated with Protein A MNase (700 ng/ml in Dig-wash buffer) for 1 h by rotation at 4° C. After three times of washes, cell pellets were resuspended in 100 μl Dig-wash buffer with 2 μl 100 mM CaCl2 and incubated at 0° C.for 30 min; reactions were stopped by addition of 100 μl 2× STOP buffer (340 mM NaCl, 20 mM EDTA pH 8.0, 4 mM EGTA, 0.05% digitonin, 50 μg/ml RNase A, 50 μg/ml glycogen). The supernatant DNA was collected after centrifugation and further purified using phenol-chloroform-isoamyl alcohol (Sigma, Cat #p3803) extraction and ethanol precipitation. Purified DNA was used for library construction using the NEBNext Ultra DNA Library Prep Kit (NEB, Cat #E7645) following the manufacturer's instructions, and the libraries were sequenced in a 75-base pair single-end run on the Next Seq 500 (Illumina) at Wistar Genomic facility.

### Bioinformatics and Statistical Analysis

RNA-Seq data was aligned using bowtie2 (32) against mm10 version of the mouse genome and RSEM v1.2.12 software was used to estimate raw read counts and FPKM values using Ensemble transcriptome information. DESeq2 (33) was used to estimate significance of differential expression between Kdm5a knockout and parental samples. Overall gene expression changes were considered significant if passed FDR<5%. CUT&RUN data was aligned using bowtie (34) against mm10 version of the mouse genome and HOMER (35) was used to call significant peaks in using “-histone” option. Genes that had a significant H3K4Me3 peak and Kdm5a binding signal of at least 4-fold over control within 500bp from TSS were considered and overlapped with genes significantly upregulated in Kdm5a knockout cells. Significance of overlap was tested using hypergeometric test using 21,588 expressed Ensemble genes as a population size. Gene set enrichment analysis of gene sets was done using GSEA Pathway Analysis (36) using “KEGG pathway database”. TCGA Agilent RNA-seq expression data for 300 HGSOC samples with copy number variation calls was downloaded from cBioPortal. Expression of CD8A, GZMB, HLA-A and HLA-B was tested for negative association with KDM5A expression. KDM5A expression data was tested for differences between samples with amplified KDM5A vs. non-amplified KDM5A using two sample Student's t-test. Statistical analyses were performed using GraphPad Prism 6 (GraphPad). Quantitative data are expressed as mean +SEM unless otherwise stated. Pearson's test was used to measure statistical correlation. For all statistical analyses, the level of significance was set at 0.05.

### Example 2—Results

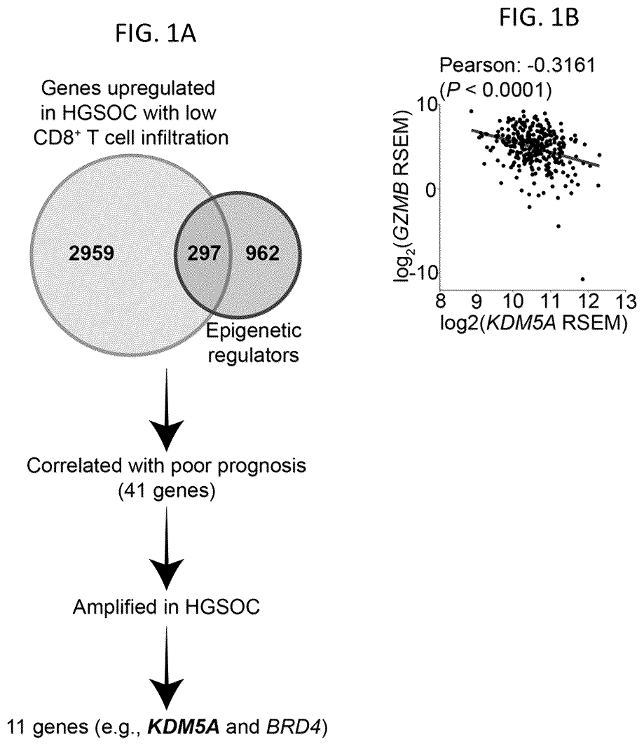

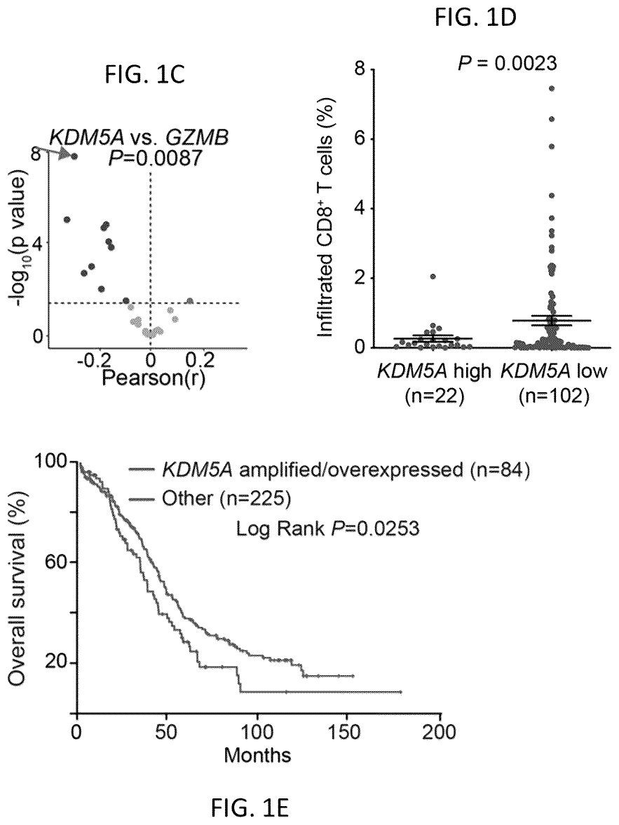

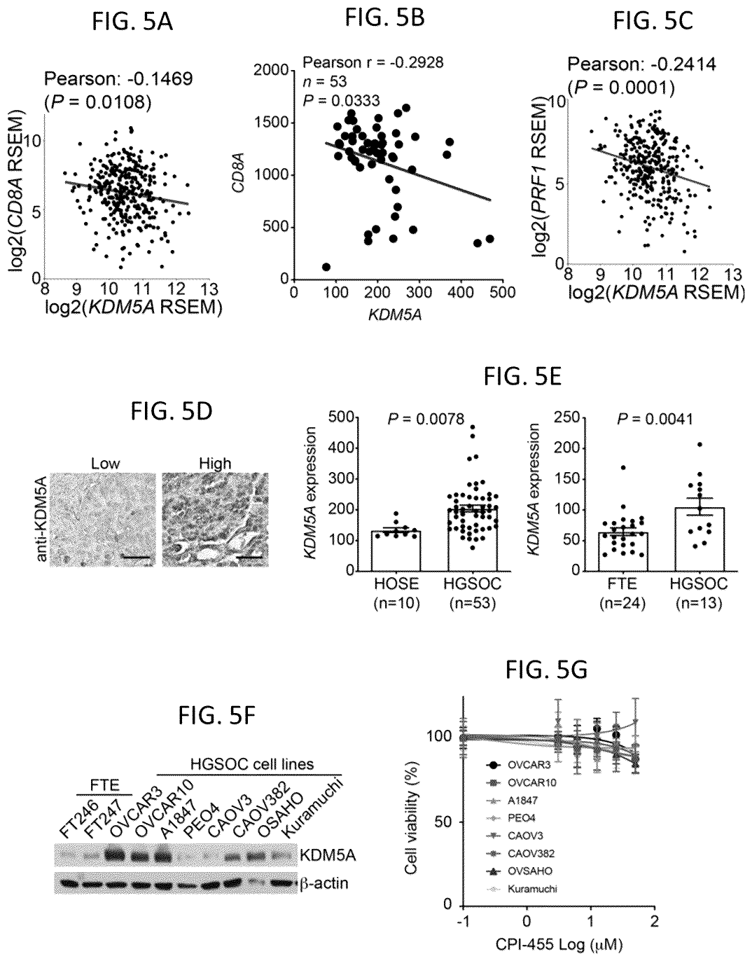

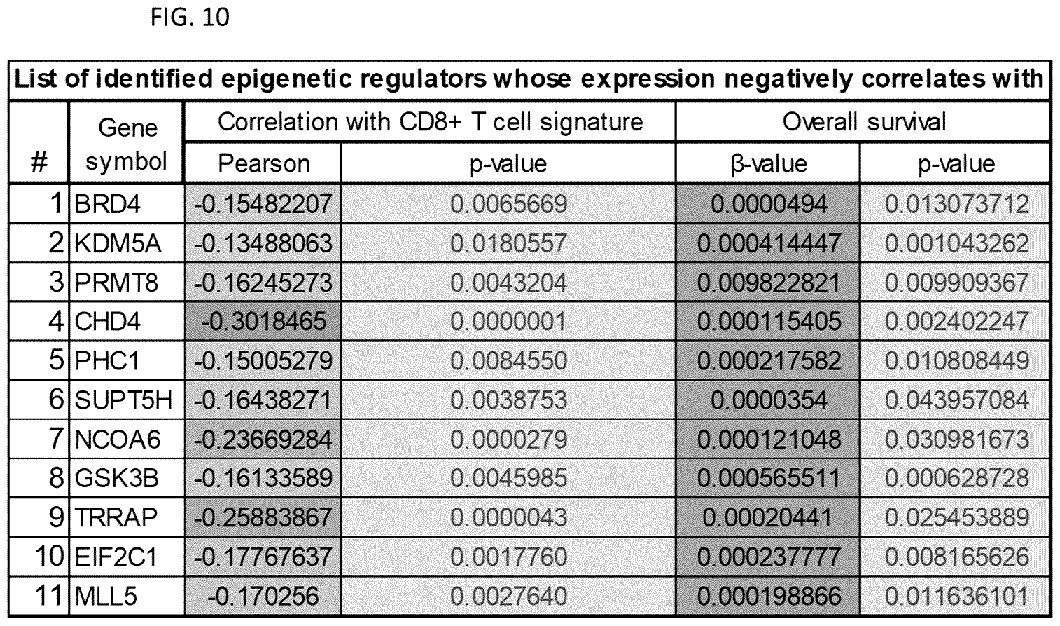

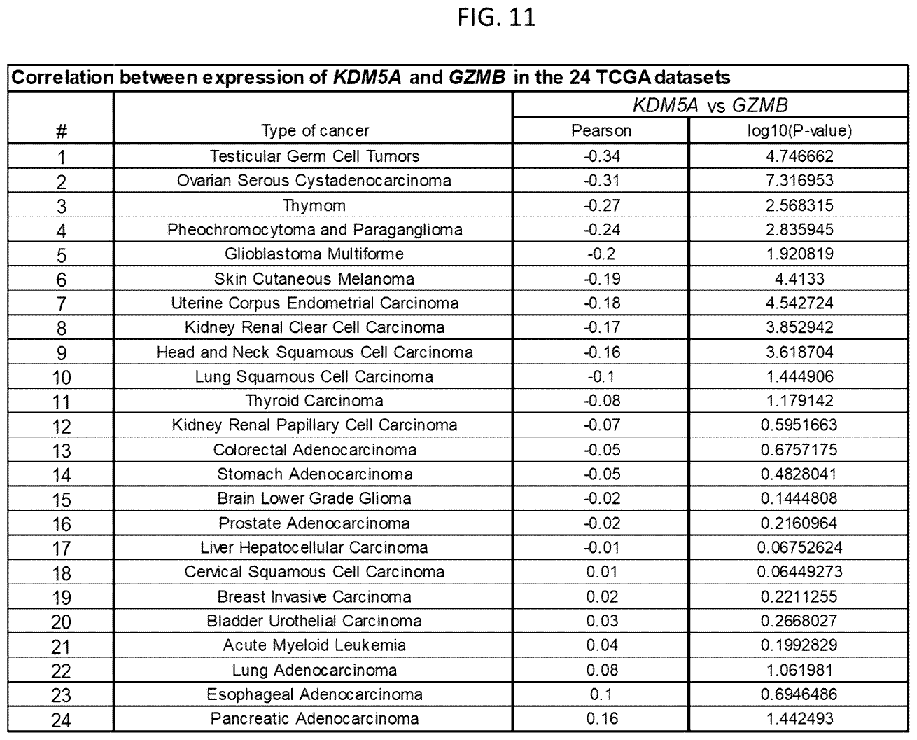

KDM5A is amplified/overexpressed in EOC and its amplification/high expression negatively correlates with the infiltration of CD8+ T cells. To identify epigenetic regulators of CD8+ T cells infiltration systematically and unbiasedly in HGSOC, we analyzed the TCGA HGSOC dataset to find epigenetic regulators whose expression was negatively correlated with infiltration of cytotoxic CD8+ T cell signature as identified by CIBERSORT (FIG. 1A and FIG. 10). This is because these epigenetic regulators may represent new therapeutic targets whose inhibition may promote infiltration of CD8+ T cells. In addition, to ensure the relevance of our findings, we prioritized the analysis for those epigenetic regulators whose high expression is negatively associated with the overall survival in HGSOCs. This analysis identified 11 genes that meet these criteria. Validating our approach, this analysis identified several genes that have been previously implicated in regulation of immune infiltration such as BRD4, CHD4, and GSK3B (FIG. 10) (39-41). KDM5A, an H3K4me3 demethylase, was the top epigenetic factor identified by this approach that has not been previously associated with the regulation of infiltration of CD8+ T cells (FIG. 5A and FIG. 10). Similarly, a negative correlation between KDM5A and CD8A expression was observed among an independent dataset consisting of 53 cases of laser-capture microdissected HGSOCs (FIG. 5B). Furthermore, KDM5A expression negatively correlated with GZMB and PRFI expression, activation markers for CD8+ T cells (FIG. 1B and FIG. 5C). Finally, a negative correlation between expression of KDM5A and GZMB was observed in a significant number of the cancer types among the 24 TCGA datasets with more than 100 patients (FIG. 1C and FIG. 11).

We next sought to determine the correlation between KDM5A protein expression and intra-tumor CD8+ T cell infiltration in a tumor microarray consisting of 124 cases of human HGSOCs. We observed that low KDM5A expression was associated with a higher intra-tumor CD8+ T cells infiltration (FIG. 1D and FIG. 5D). Low level of CD8+ T cell infiltration is a major factor associated with poor survival in HGSOCs (42-44). Consistently, we observed that high KDM5A expression is associated with poor prognosis in the TCGA HGSOC dataset (FIG. 1E). Notably, KDM5A is upregulated in HGSOCs comparing to normal tissues from laser-capture microdissections (FIG. 5E). Additionally, KDM5A expression was also found to be upregulated in 75% (6/8) EOC cell lines tested compared with fallopian tube epithelial cells (FIG. 5F). We conclude that high KDM5A expression negatively correlates with CD8+ T cell infiltration and predicts poor survival in HGSOCs. We next sought to determine whether inhibition of KDM5A activity affects the growth of EOC cells. Toward this goal, we treated the panel of EOC cell lines with various degree of KDM5A expression with CPI-455, a small molecule inhibitor of KDM5A (45). Consistently with previous studies (46), CPI-455 did not affect cell viability in vitro regardless of KDM5A expression status in a dose response curve analysis (FIG. 5G). Thus, we conclude that KDM5A inhibition does not affect the growth of EOC cells. KDM5A inhibition promotes anti-tumor immune response in vivo.

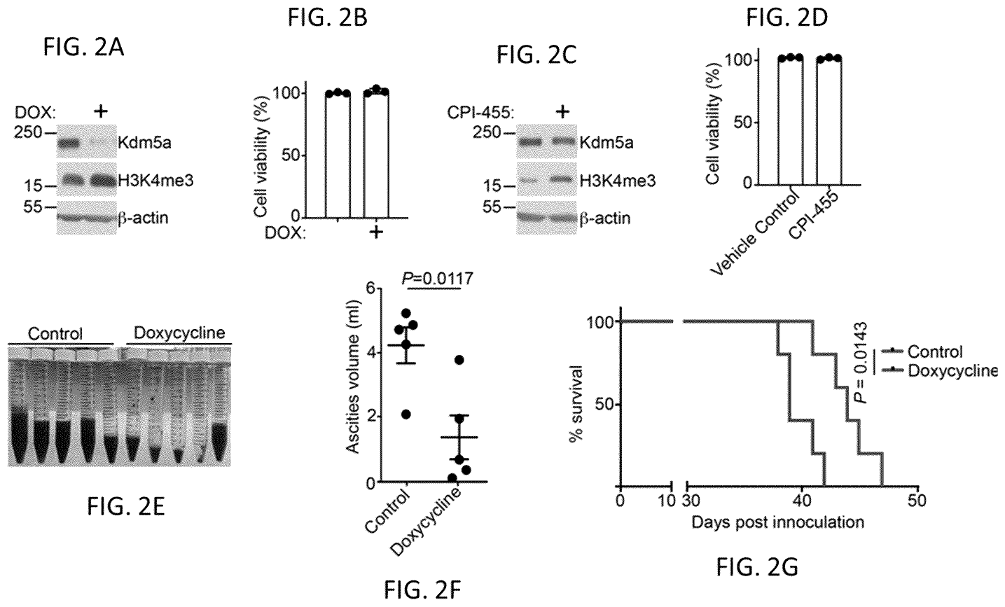

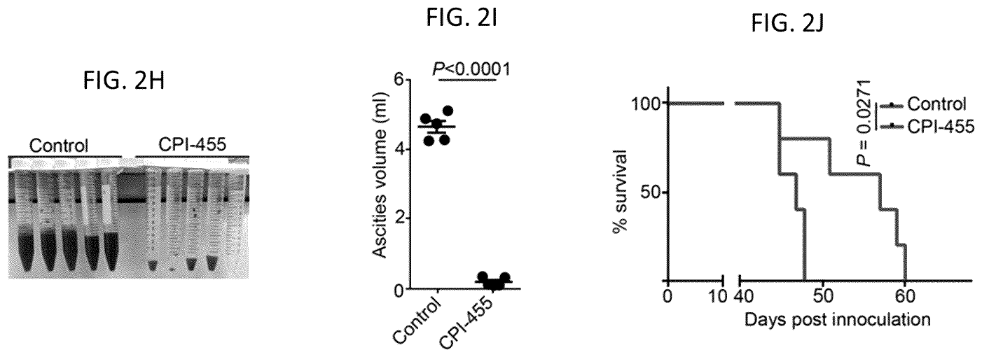

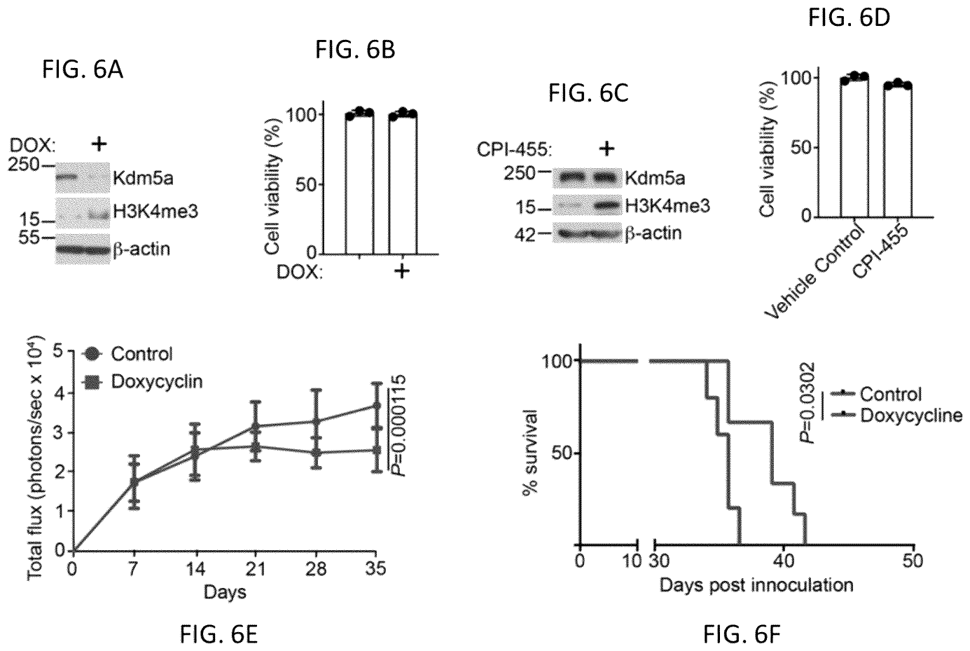

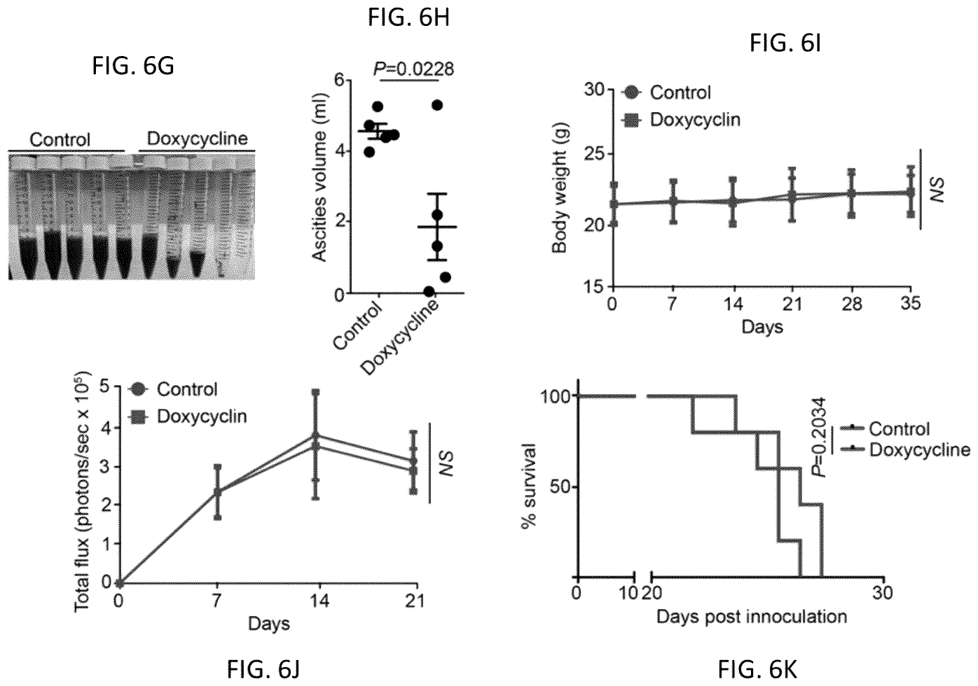

Since KDM5A inhibition does not affect the viability of EOC cells, we sought to determine whether KDM5A contributes to EOC through tumor immune microenvironment. Toward this goal, we developed a doxycycline-inducible shRNA-mediated Kdm5a-knockdown mouse EOC cell line ID8. As expected, induced Kdm5a depletion led to increased levels of H3K4me3, the substrate of Kdm5a (FIG. 2A). Consistent with the notion that KDM5A inhibitor failed to affect the viability of human EOC cell lines regardless of KDM5A expression levels, shRNA mediated Kdm5a knockdown did not affect the growth of ID8 cells in vitro. (FIG. 2B). Similar observations were made in two independent ID8 inducible shKdm5a clones (FIG. 2A - FIG. 2B and FIG. 6A-FIG. 6B). Likewise, a small molecule KDM5A inhibitor CPI-455 did not affect cell viability, despite its ability to increase H3K4me3 levels in two mouse EOC cell lines ID8 and HGS2 (FIG. 2C-FIG. 2D and FIG. 6C-FIG. 6D). To determine the effects of KDM5A inhibition on the growth of KDM5A-expressing EOC in vivo, we utilized a syngeneic immunocompetent EOC mouse model. Specifically, immunocompetent C57BL/6 mice were injected intraperitonially (i.p.) with a luciferase-expressing doxycycline-inducible shKdm5a-expressing mouse EOC ID8 cells. Cells were allowed to grow for one week and then mice were randomized based on luciferase signal. Mice were next treated with or without doxycycline containing food as previously reported (47). Doxycycline treatment significantly reduced the tumor burden as measured by luminescence, which correlated with an improvement of the survival of tumor bearing mice in the immunocompetent C57BL/6 syngeneic ID8 mouse EOC model established by two independent inducible clones (FIG. 2E-FIG. 2G and FIG. 6E-FIG. 6F). Notably, doxycycline treatment also significantly reduced amount of ascites produced, a key feature of EOC clinical presentation (FIG. 2E-FIG. 2F and FIG. 6G-FIG. 6H) (48). Also, doxycycline treatment did not affect the body weight of tumor bearing mice (FIG. 6I). As a control, doxycycline treatment did not significantly inhibit tumor cell growth in vivo or affect the survival of tumor bearing mice established in parallel in immune-compromised NSG mice (FIG. 6J-FIG. 6K). Together, we conclude that Kdm5a knockdown reduces tumor burden and improves the survival of tumor-bearing mice in immune-competent but not in immune-compromised mouse EOC model.

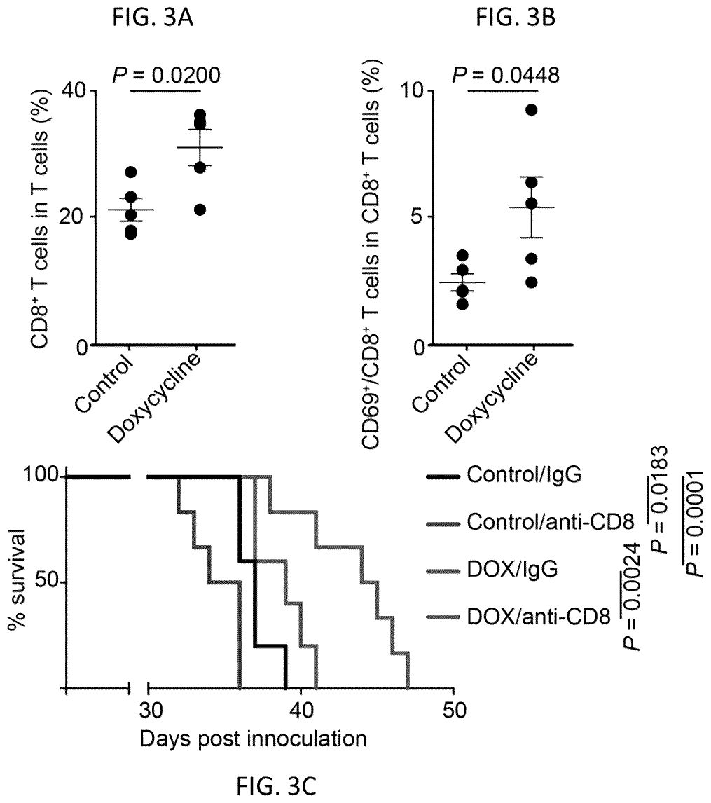

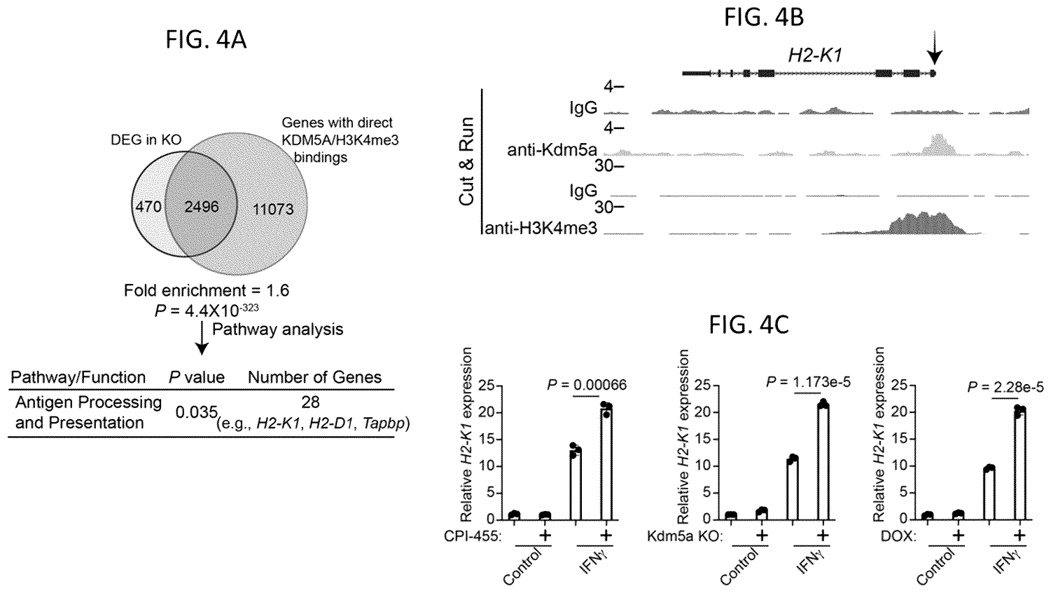

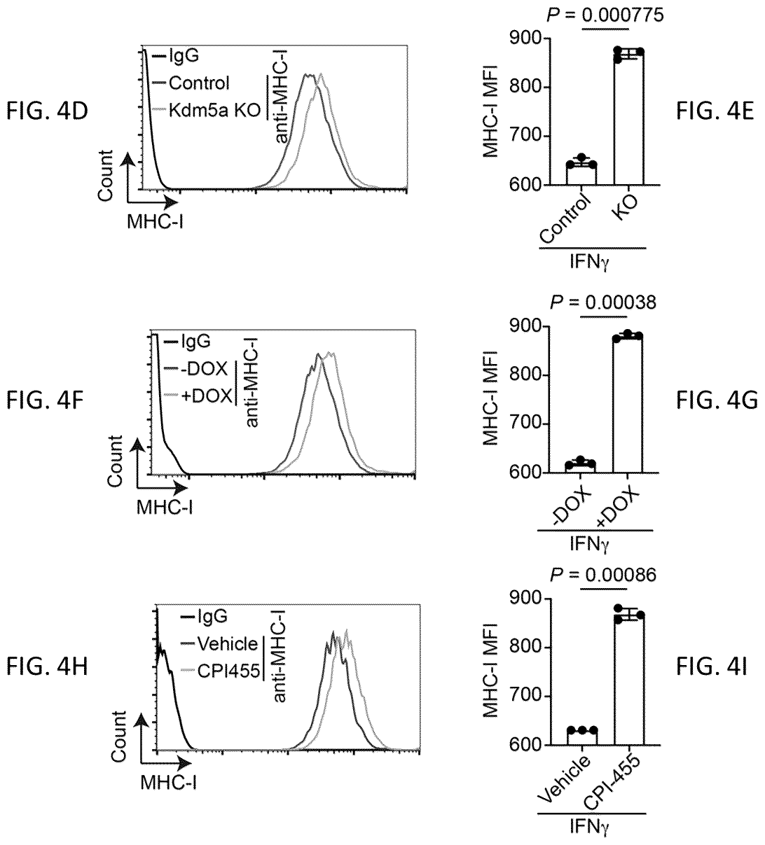

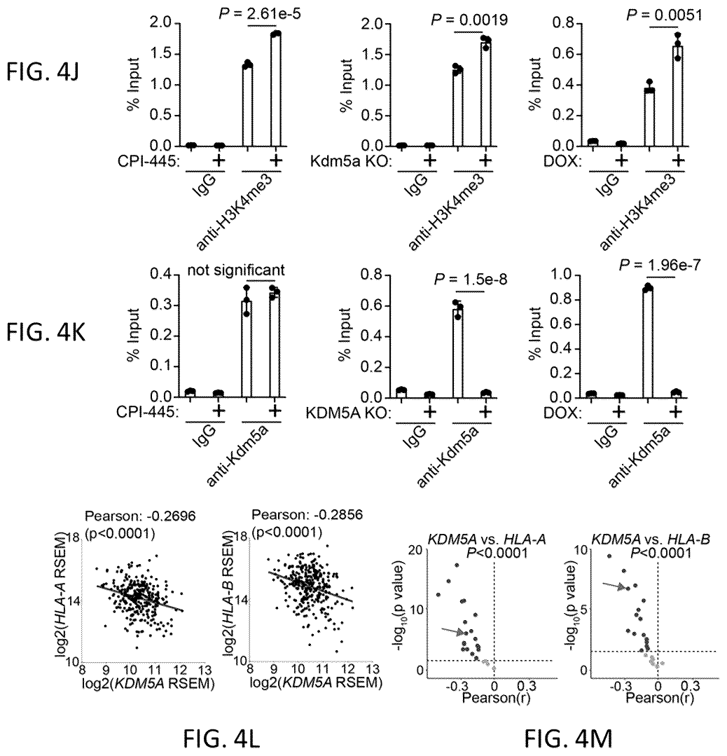

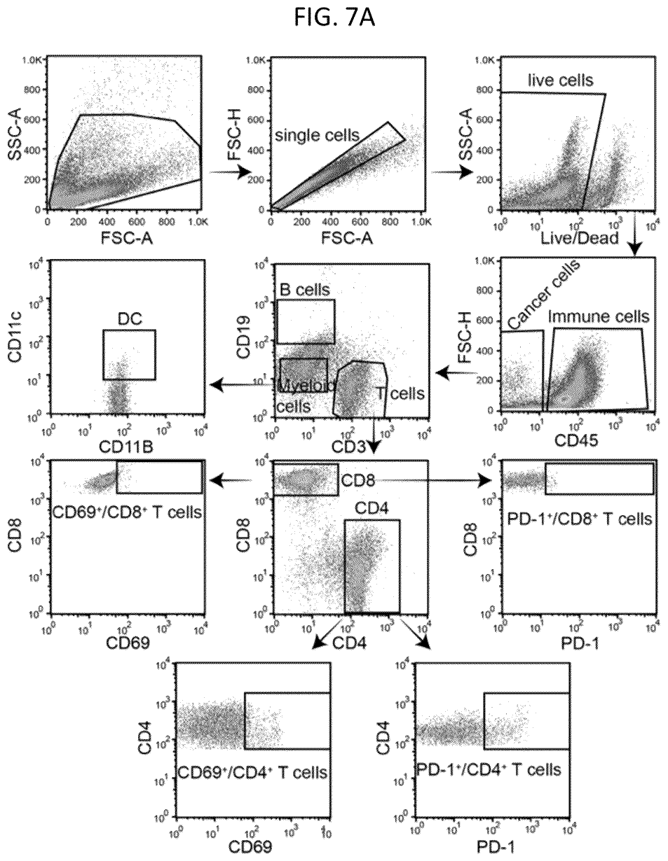

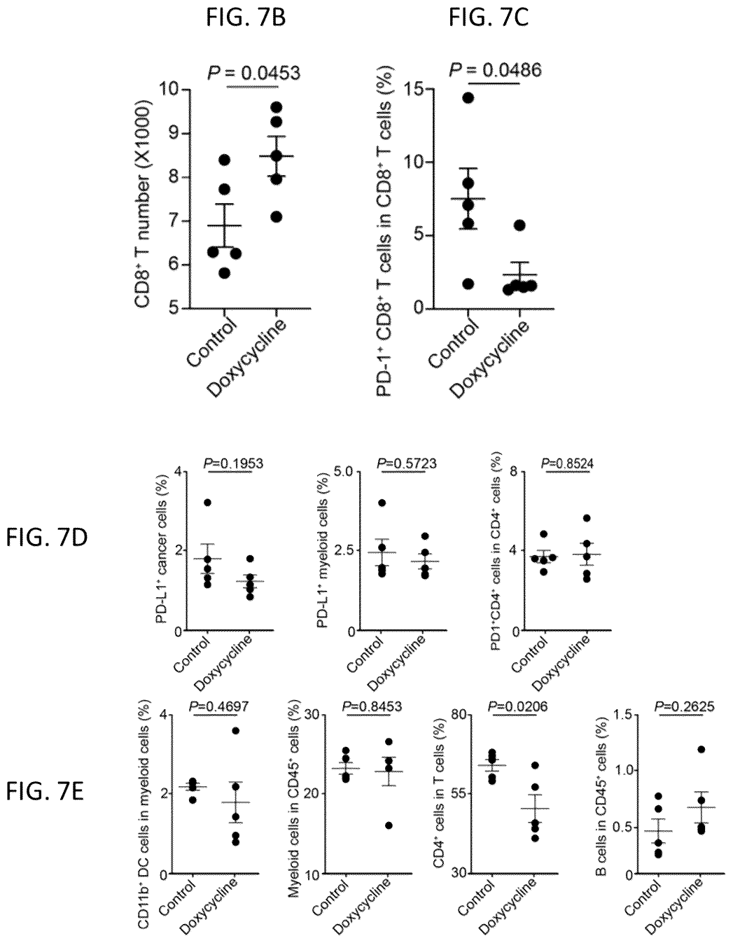

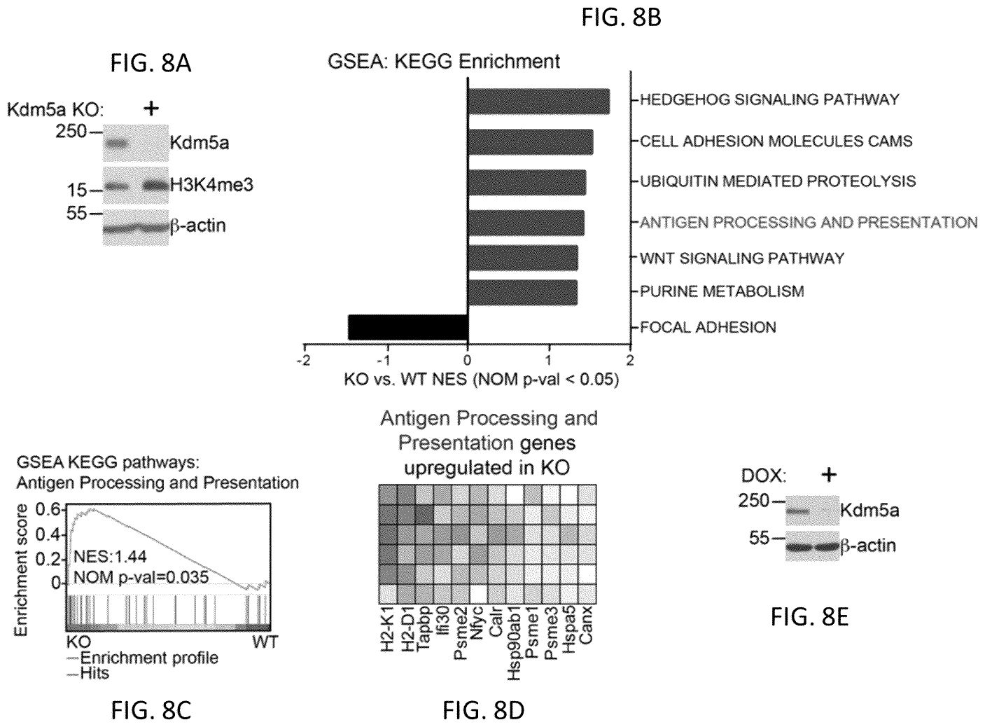

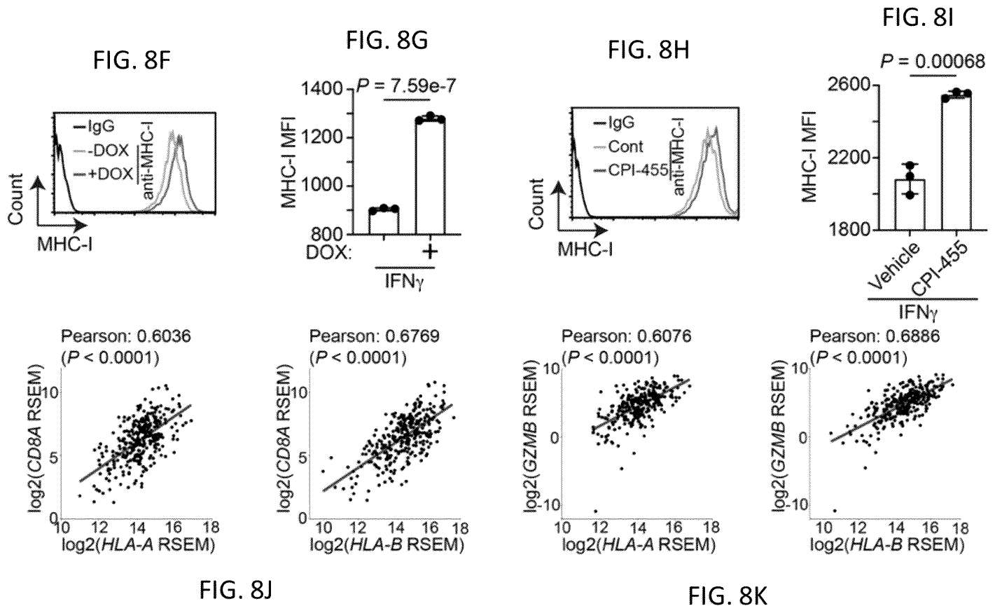

We next sought to complement the genetic knockdown in vivo using KDM5A inhibitor CPI-455. Towards this goal, ID8 cells were first injected i.p. in immuno-competent C57BL/6 mice and the cells were allowed to grow for one week to establish the tumors. Mice were then randomized and treated daily with vehicle control or CPI-455 (50 mg/kg) by i.p. injection. Similar to Kdm5a knockdown, CPI-455 treatment significantly reduced ascites production and improved the survival of tumor bearing mice (FIG. 2H-FIG. 2J). Thus, we conclude that the antitumor effect of KDM5A inhibition depends on immune microenvironment. Anti-tumor immune response mediated by KDM5A inhibition depends on CD8+ T cells. To test our hypothesis that KDM5A inhibition reduces tumor growth through regulation of immune infiltration, we profiled changes in immune cells in ascites collected from mice bearing doxycycline-inducible shKdm5a ID8 cells in the presence or absence of doxycycline (FIG. 7A). Consistent with our CIBERSORT analysis, we have observed an increase in the percentage and total number of CD8+ T cells in mice with depleted Kdm5a levels (FIG. 3A and FIG. 7B). In addition to increased total number of CD8+ T cells, we also observed that Kdm5a inhibition resulted in an increase in the percentage of activated CD8+ T cells that are marked by CD69+expression (FIG. 3B). Previous studies have demonstrated that KDM5A potentially regulates immune response through altering the immune checkpoint pathways (49). Accordingly, we tested PD-L1 expression on the surface of tumor cells and CD11B+ myeloid cells as well as PD-1 expression on the surface of CD4+ T and CD8+ T cells isolated from ascites. Indeed, we observed that Kdm5a depletion led to a decrease in PD-1+/CD8+ T cells (FIG. 7C). However, we did not observe any significant effect of Kdm5a depletion on PD-L1 expression in either cancer or myeloid cells (FIG. 7D). Likewise, we also didn't observe any changes in PD-1 expression on CD4+ T cells (FIG. 7D). In contrast to CD8+ T cells, percentage of other immune cell types such as dendritic cells, myeloid cells, and B-cells did not increase in ascites collected from mice bearing Kdm5a-depleted ID8 cells (FIG. 7E). Infiltration of cytotoxic CD8+ T cells plays a critical role in promoting anti-tumor immune response. Thus, we next sought to investigate whether the anti-tumor effects promoted by Kdm5a inhibition depend on CD8+ T cells. Toward this goal, we depleted CD8+ T cells with an anti-CD8 antibody in immune-competent C57BL/6 mice bearing doxycycline-inducible shKdm5a ID8 cells in the presence or absence of doxycycline. Indeed, we observed that anti-CD8 antibody treatment abrogated the anti-tumor effect of Kdm5a inhibition in vivo as evidenced by the loss of improvement of survival of tumor bearing mice (FIG. 3C). Thus, our data demonstrate that CD8+ T cells mediate the anti-tumor immune effects induced by KDM5A inhibition. KDM5A regulates antigen processing and presentation pathway. To explore the mechanistic basis of Kdm5a-regulated infiltration of CD8+ T cells, we performed RNA-seq in parental control and Kdm5a-knockout ID8 cells. Since KDM5A functions as a suppressor of gene expression (17), we focused our analysis on genes that are upregulated by Kdm5a knockout. This analysis revealed a list of 2966 genes that were significantly differentially expressed (FDR<5%) by Kdm5a knockout in ID8 cells comparing to controls (FIG. 4A and FIG. 8A). To identify direct Kdm5a target genes, we performed CUT&RUN for Kdm5a and its enzymatic substrate H3K4me3 in control and Kdm5a knockout ID8 cells (FIG. 4A). Cross-referencing RNA-seq and CUT&RUN data revealed a list of 2496 direct Kdm5a target genes that were differentially expressed in Kdm5a knockout ID8 cells (FIG. 4A). This represents a significant overlap (1.6-fold over expected by chance, P=4.4×10⁻³²³) between 11073 genes that were differentially expressed in Kdm5a knockout cells comparing to control ID8 cells and genes that were occupied by Kdm5a/H3K4me3 (FIG. 4A). Pathway analysis revealed that one of the top functional pathways enriched by Kdm5a knockout cells is antigen processing and presentation pathway (P=0.035) (FIG. 4A and FIG. 8C-FIG. 8C). Notably, the three top ranked genes (namely H2-K1, H2-D1, and Tapbp1) in this list all encode key proteins involved in MHC class I-mediated antigen presentation (FIG. 4A and FIG. 8D). MHC class I antigen presentation pathway inhibition has been shown to be one of the major mechanisms underlying poor immune cell infiltration (8). One of the roles of MHC class I complex is to present cancer cell antigens to cytotoxic CD8+ T cells. H2-K1 (known in human genome as HLA-A) is a component of MHC class I complex and its decreased expression on the surface of cancer cells results in reduced immune infiltration and impaired anti-tumor immune response (12-14). Notably, CUT&RUN revealed that Kdm5a and H3K4me3 are enriched at the promoter of the mouse H2-Kl gene (FIG. 4B).

We first sought to validate upregulation of H2-K1 expression by Kdm5a inhibition in response to IFNγ treatment, which is known to promote expression of MHC class I genes to regulate the anti-tumor immune response (50). Specifically, the expression of H2-K1 was upregulated by KDM5A inhibitor CPI-455 treatment (FIG. 4C). Likewise, Kdm5a knockout or doxycycline-inducible knockdown also increased the expression of H2-K1 in response to IFNγ treatment (FIG. 4C). Kdm5a knockout or knockdown increased MHC class I presence on the surface of ID8 and HGS2 cells in response to IFNγstimulation (FIG. 4D-FIG. 4G and FIG. 8E-FIG. 8G). Similarly, CPI-455 treatment increased MHC class I presence on cell surface in response to IFNγ treatment (FIG. 4H-FIG. 4I and FIG. 8H-FIG. 8I). We next validated changes in the association of H3K4me3 with the H2-K1 gene promoter upon Kdm5a inhibition (FIG. 4J), which inversely correlated with the changes in its association with Kdm5a (FIG. 4K). Notably, CPI-455 increased the association of H3K4me3, but not Kdm5a, with H2-K1 gene promoter (FIG. 4J-FIG. 4K), which is consistent with the notion that CPI-455 inhibits the enzymatic activity of KDM5A instead of its association with its target genes.

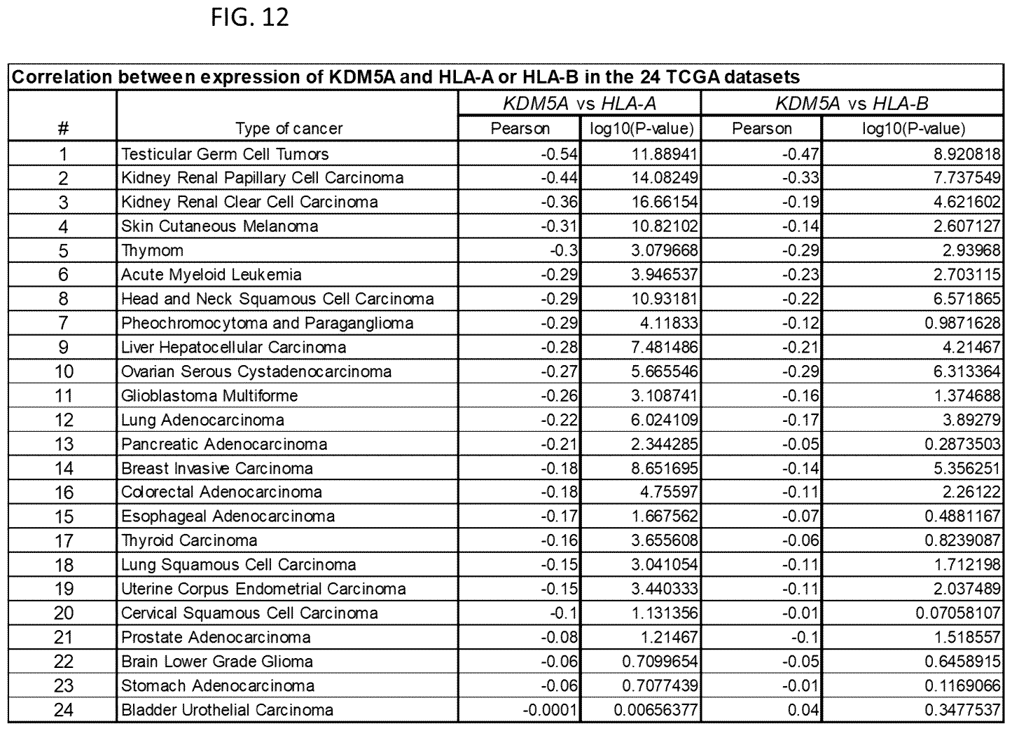

To further support our observation that KDM5A regulates the expression of MHC class I complex we explored the TCGA HGSOC dataset. Indeed, in HGSOC KDM5A negatively correlated with two major subunits of human MHC class I complex, HLA-A and HLA-B (FIG. 4L). Notably, a negative correlation between KDM5A expression and HLA-A or HLA-B expression was observed in a statistically significant majority of the cancer types in the 24 TCGA datasets with more than 100 patients (FIG. 4M and FIG. 12). Additionally, HLA-A and HLA-B expression highly correlates with expression of CD8A as well as GZMB in the TCGA HGSOC dataset (FIG. 8J-FIG. 8K). Together, our data supports that KDM5A exhibits its oncogenic function through downregulation of antigen processing and presentation machinery such as MHC class I complex.

### Example 3

Our data demonstrates that KDM5A regulates anti-tumor immune response. Mechanistically, KDM5A suppresses the expression of genes involved in antigen processing and presentation pathway. Consistently, impairment in antigen processing and presentation is observed in different types of cancers and often leads to immune surveillance evasion and low levels of immune infiltration (8,9). Specifically, KDM5A regulates expression of MHC class I pathway genes such as H2-K1, which correlated with a decreased presence of MHC class I complex on surface of cancer cells. MHC class I complex is responsible for tumor antigen presentation to cytotoxic CD8+ T cells and low level of MHC class I expression is associated with reduced immune infiltration in several types of cancer (12-14). However, we cannot rule out the possibility that other mechanisms such as upregulation of chemokines induced by KDM5A inhibition may also contribute to the observed changes in infiltration of CD8+ T cells. Notably, expression of antigen presentation genes repressed by Kdm5a could be restored by small molecule inhibitors such as CPI-455. However, a recent study showed that in a melanoma mouse model, the reduced expression of MHC class I expression does not affect CD8+ T cell infiltration (51). This suggests that the role of MHC class I in regulating CD8+ T cell infiltration may be cancer type and/or context dependent. Regardless, our in vivo studies demonstrated that Kdm5a inhibition resulted in increased CD8+ T cell infiltration, decreased ascites formations, and improved survival of tumor-bearing mice. KDM5A plays a context dependent role in cancer. Despite the evidence supporting an overall oncogenic role of KDM5A in several types of cancer (15,16), we and other groups did not observe decreased cell viability upon KDM5A inhibition in vitro (27). Likewise, we show here that Kdm5a inhibition failed to significantly suppress tumor growth in immunocompromised mouse models. However, multiple studies have demonstrated that KDM5A may promote cancer in vivo through regulating cell cycle, invasion, epithelial-mesenchymal transition and chemoresistance (15,16,20-23). Regardless, our results support that KDM5A inhibition could potentially be a viable cancer therapeutic strategy through boosting anti-tumor immunity. Several studies have recently demonstrated the role of KDM5A and other KDM5 family members in the regulation of a tumor immune response. For example, increased expression of KDM5A improves the response to immune checkpoint blockade in a melanoma mouse model, which was attributed to an overall higher recruitment of CD8+ T cells due to changes in the myeloid compartment in a melanoma model (49). However, our data showed that KDM5A inhibition promotes CD8+ T cells and we did not observe a significant change in myeloid cells, indicating that KDM5A's role in regulating tumor immune microenvironment may be cancer type and/or context dependent. Other members of KDM5 family such as KDM5B, but not KDM5A, were shown to repress the immune response via suppression of STING pathway (52). KDM5B also promotes immune evasion by epigenetic silencing of transposable elements (53). Together with the present study, these findings support the notion that KDM5 family proteins play a role in regulating tumor immune response. Immune surveillance evasion is a major obstacle to overcome in developing effective EOC immunotherapy and our present study suggests KDM5A may play a role in this process. Therefore, the results presented here serve as a scientific rationale for targeting KDM5A overexpressed EOCs, which represents ˜26% of HGSOCs. Notably, several KDM5A or panKDM5 inhibitors has been recently developed and are currently under various stages of preclinical evaluations (27). In addition to HGSOC, our results show that KDM5A expression negatively correlates with the expression of antigen presentation genes in the majority of cancer types. In summary, our results demonstrate that targeting KDM5A demethylase activity represents a viable therapeutic strategy to boost anti-tumor immunity.

### REFERENCES

1.Fridman WH, Zitvogel L, Sautes-Fridman C, Kroemer G. The immune contexture in cancer prognosis and treatment. Nat Rev Clin Oncol 2017; 14:717-34
2.Fridman WH, Galon J, Dieu-Nosjean MC, Cremer I, Fisson S, Damotte D, et al. Immune infiltration in human cancer: prognostic significance and disease control. Curr Top Microbiol Immunol 2011;344:1-24
3.Bruni D, Angell HK, Galon J. The immune contexture and Immunoscore in cancer prognosis and therapeutic efficacy. Nat Rev Cancer 2020;20:662-80
4. Topper MJ, Vaz M, Chiappinelli KB, DeStefano Shields CE, Niknafs N, Yen RC, et al. Epigenetic Therapy Ties MYC Depletion to Reversing Immune Evasion and Treating Lung Cancer. Cell 2017; 171:1284-300 e21
5.Dunn J, Rao S. Epigenetics and immunotherapy: The current state of play. Mol Immunol 2017;87:227-39
6.Cao J, Yan Q. Cancer Epigenetics, Tumor Immunity, and Immunotherapy. Trends Cancer 2020;6:580-92
7.Hogg SJ, Beavis PA, Dawson MA, Johnstone RW. Targeting the epigenetic regulation of antitumour immunity.
8.Nat Rev Drug Discov 2020; 19:776-800 Jhunjhunwala S, Hammer C, Delamarre L. Antigen presentation in cancer: insights into tumour immunogenicity and immune evasion. Nat Rev Cancer 2021;21:298-312
9.Garrido F, Algarra I, Garcia-Lora AM. The escape of cancer from T lymphocytes: immunoselection of MHC class I loss variants harboring structural-irreversible “hard” lesions. Cancer Immunol Immunother 2010;59:1601-6
10. Wieczorek M, Abualrous ET, Sticht J, Alvaro-Benito M, Stolzenberg S, Noe F, et al. Major Histocompatibility Complex (MHC) Class I and MHC Class II Proteins: Conformational Plasticity in Antigen Presentation. Front Immunol 2017;8:292
11. Vesely MD, Kershaw MH, Schreiber RD, Smyth MJ. Natural innate and adaptive immunity to cancer. Annu Rev Immunol 2011;29:235-71
12. Dhatchinamoorthy K, Colbert JD, Rock KL. Cancer Immune Evasion Through Loss of MHC Class I Antigen Presentation. Front Immunol 2021;12:636568
13. Dersh D, Holly J, Yewdell JW. A few good peptides: MHC class I-based cancer immunosurveillance and immunoevasion. Nat Rev Immunol 2021;21:116-28
14. Burr ML, Sparbier CE, Chan KL, Chan YC, Kersbergen A, Lam EYN, et al. An Evolutionarily Conserved Function of Polycomb Silences the MHC Class I Antigen Presentation Pathway and Enables Immune Evasion in Cancer. Cancer Cell 2019;36:385-401 e8
15. Yang GJ, Zhu MH, Lu XJ, Liu YJ, Lu JF, Leung CH, et al. The emerging role of KDM5A in human cancer. J Hematol Oncol 2021; 14:30
16. Plch J, Hrabeta J, Eckschlager T. KDM5 demethylases and their role in cancer cell chemoresistance. Int J Cancer 2019; 144:221-31
17. Christensen J, Agger K, Cloos PA, Pasini D, Rose S, Sennels L, et al. RBP2 belongs to a family of demethylases, specific for tri-and dimethylated lysine 4 on histone 3. Cell 2007;128:1063-76
18. Shilatifard A. Molecular implementation and physiological roles for histone H3 lysine 4 (H3K4) methylation. Curr Opin Cell Biol 2008;20:341-8
19. Howe FS, Fischl H, Murray SC, Mellor J. Is H3K4me3 instructive for transcription activation? Bioessays 2017;39:1-12
20. Beshiri ML, Holmes KB, Richter WF, Hess S, Islam AB, Yan Q, et al. Coordinated repression of cell cycle genes by KDM5A and E2F4 during differentiation. Proc Natl Acad Sci U S A 2012; 109:18499-504
21. Dai B, Huang H, Guan F, Zhu G, Xiao Z, Mao B, et al. Histone demethylase KDM5A inhibits glioma cells migration and invasion by down regulating ZEB1. Biomed Pharmacother 2018;99:72-80 22. 23. 24. 25. 26. 27. 28. 29.
22. Feng T, Wang Y, Lang Y, Zhang Y. KDM5A promotes proliferation and EMT in ovarian cancer and closely correlates with PTX resistance. Mol Med Rep 2017;16:3573-80
23. Banelli B, Carra E, Barbieri F, Wurth R, Parodi F, Pattarozzi A, et al. The histone demethylase KDM5A is a key factor for the resistance to temozolomide in glioblastoma. Cell Cycle 2015; 14:3418-29
24. Nie Z, Shi L, Lai C, O'Connell SM, Xu J, Stansfield RK, et al. Structure-based design and discovery of potent and selective KDM5 inhibitors. Bioorg Med Chem Lett 2018;28:14904
25. Horton JR, Liu X, Gale M, Wu L, Shanks JR, Zhang X, et al. Structural Basis for KDM5A Histone Lysine Demethylase Inhibition by Diverse Compounds. Cell Chem Biol 2016;23:769-81
26. Gehling VS, Bellon SF, Harmange JC, LeBlanc Y, Poy F, Odate S, et al. Identification of potent, selective KDM5 inhibitors. Bioorg Med Chem Lett 2016;26:4350-4
27. Yang GJ, Wu J, Miao L, Zhu MH, Zhou QJ, Lu XJ, et al. Pharmacological inhibition of KDM5A for cancer treatment. Eur J Med Chem 2021;226:113855
28. Lin J, Liu H, Fukumoto T, Zundell J, Yan Q, Tang CA, et al. Targeting the IRE1alpha/XBP1s pathway suppresses CARM1-expressing ovarian cancer. Nat Commun 2021;12:5321
29. Jordan KR, Sikora MJ, Slansky JE, Minic A, Richer JK, Moroney MR, et al. The Capacity of the Ovarian Cancer Tumor Microenvironment to Integrate Inflammation Signaling Conveys a Shorter Disease-free Interval. Clin Cancer Res 2020;26:6362-73
30. Watson ZL, Yamamoto TM, McMellen A, Kim H, Hughes CJ, Wheeler LJ, et al. Histone methyltransferases EHMT1 and EHMT2 (GLP/G9A) maintain PARP inhibitor resistance in high-grade serous ovarian carcinoma. Clin Epigenetics 2019;11:165
31. Chen B, Khodadoust MS, Liu CL, Newman AM, Alizadeh AA. Profiling Tumor Infiltrating Immune Cells with CIBERSORT. Methods Mol Biol 2018; 1711:243-59
32. Langmead B, Salzberg SL. Fast gapped-read alignment with Bowtie 2. Nat Methods 2012;9:357-9
33. Li B, Dewey CN. RSEM: accurate transcript quantification from RNA-Seq data with or without a reference genome. BMC Bioinformatics 2011; 12:323
34. Langmead B, Trapnell C, Pop M, Salzberg SL. Ultrafast and memory-efficient alignment of short DNA sequences to the human genome. Genome Biol 2009;10:R25
35. Heinz S, Benner C, Spann N, Bertolino E, Lin YC, Laslo P, et al. Simple combinations of lineage-determining transcription factors prime cis-regulatory elements required for macrophage and B cell identities. Mol Cell 2010;38:576-89
36. Subramanian A, Tamayo P, Mootha VK, Mukherjee S, Ebert BL, Gillette MA, et al. Gene set enrichment analysis: a knowledge-based approach for interpreting genome-wide expression profiles. Proc Natl Acad Sci U S A 2005; 102:15545-50
37. Mok SC, Bonome T, Vathipadiekal V, Bell A, Johnson ME, Wong KK, et al. A gene signature predictive for outcome in advanced ovarian cancer identifies a survival factor: microfibril-associated glycoprotein 2. Cancer Cell 2009;16:521-32
38. Tone AA, Begley H, Sharma M, Murphy J, Rosen B, Brown TJ, et al. Gene expression profiles of luteal phase fallopian tube epithelium from BRCA mutation carriers resemble high-grade serous carcinoma. Clin Cancer Res 2008;14:4067-78
39. Augello G, Emma MR, Cusimano A, Azzolina A, Montalto G, McCubrey JA, et al. The Role of GSK-3 in Cancer Immunotherapy: GSK-3 Inhibitors as a New Frontier in Cancer Treatment. Cells 2020;9
40. Zhong L, Yang Z, Lei D, Li L, Song S, Cao D, et al. Bromodomain 4 is a potent prognostic marker associated with immune cell infiltration in breast cancer. Basic Clin Pharmacol Toxicol 2021; 128:169-82
41. Shao S, Cao H, Wang Z, Zhou D, Wu C, Wang S, et al. CHD4/NuRD complex regulates complement gene expression and correlates with CD8 T cell infiltration in human hepatocellular carcinoma. Clin Epigenetics 2020; 12:31
42. Hwang WT, Adams SF, Tahirovic E, Hagemann IS, Coukos G. Prognostic significance of tumor-infiltrating T cells in ovarian cancer: a meta-analysis. Gynecol Oncol 2012;124:192-8
43. Bachmayr-Heyda A, Aust S, Heinze G, Polterauer S, Grimm C, Braicu EI, et al. Prognostic impact of tumor infiltrating CD8+ T cells in association with cell proliferation in ovarian cancer patients—a study of the OVCAD consortium. BMC Cancer 2013; 13:422
44. Ovarian Tumor Tissue Analysis C, Goode EL, Block MS, Kalli KR, Vierkant RA, Chen W, et al. Dose-Response Association of CD8+ Tumor-Infiltrating Lymphocytes and Survival Time in High-Grade Serous Ovarian Cancer. JAMA Oncol 2017;3:e173290
45. Vinogradova M, Gehling VS, Gustafson A, Arora S, Tindell CA, Wilson C, et al. An inhibitor of KDM5 demethylases reduces survival of drug-tolerant cancer cells. Nat Chem Biol 2016;12:531-8
46. Leadem BR, Kagiampakis I, Wilson C, Cheung TK, Arnott D, Trojer P, et al. A KDM5 Inhibitor Increases Global H3K4 Trimethylation Occupancy and Enhances the Biological Efficacy of 5-Aza-2′-Deoxycytidine. Cancer Res 2018;78:1127-39
47. Du W, Zhang L, Brett-Morris A, Aguila B, Kerner J, Hoppel CL, et al. HIF drives lipid deposition and cancer in ccRCC via repression of fatty acid metabolism. Nat Commun 2017;8:1769
48. Kipps E, Tan DS, Kaye SB. Meeting the challenge of ascites in ovarian cancer: new avenues for therapy and research. Nat Rev Cancer 2013;13:273-82
49. Wang L, Gao Y, Zhang G, Li D, Wang Z, Zhang J, et al. Enhancing KDM5A and TLR activity improves the response to immune checkpoint blockade. Sci Transl Med 2020;12 50.51.52. 53.
50. Castro F, Cardoso AP, Goncalves RM, Serre K, Oliveira MJ. Interferon-Gamma at the Crossroads of Tumor Immune Surveillance or Evasion. Front Immunol 2018;9:847
51. Moustaki A, Crawford JC, Alli S, Fan Y, Boi S, Zamora AE, et al. Antigen crosspresentation in young tumor-bearing hosts promotes CD8(+) T cell terminal differentiation. Sci Immunol 2022;7:eabf6136
52. Wu L, Cao J, Cai WL, Lang SM, Horton JR, Jansen DJ, et al. KDM5 histone demethylases repress immune response via suppression of STING. PLOS Biol 2018; 16:e2006134.
53. Zhang SM, Cai WL, Liu X, Thakral D, Luo J, Chan LH, et al. KDM5B promotes immune evasion by recruiting SETDB1 to silence retroelements. Nature 2021;598:682-7.

All patents, patent publications, and other publications listed in this specification are incorporated herein by reference. U.S. Provisional Patent Application No. 63/378,895, filed Oct. 10, 2022, is incorporated by reference. While the invention has been described with reference to a particularly preferred embodiment, it will be appreciated that modifications can be made without departing from the spirit of the invention. Such modifications are intended to fall within the scope of the appended claims.

What is claimed is:

1. A method of increasing intratumor CD8+ T-cell infiltration in a subject in need thereof, the method comprising administering a KDM5A inhibitor to the subject.

2. A method of augmenting tumor antigen processing and/or presentation in a subject in need thereof, the method comprising administering a KDM5A inhibitor to the subject.

3. A method of treating cancer in a subject in need thereof, the method comprising administering a KDM5A inhibitor and i) a cytotoxic agent, ii) interferon gamma, and/or iii) a checkpoint inhibitor to the subject.

4. The method of claim 1, wherein the subject has ovarian cancer.

5. The method of claim 1, wherein the subject has high-grade serous ovarian cancer (HGSOC).

6. The method of claim 1, wherein the subject has triple negative breast cancer.

7. The method of claim 1, wherein the KDM5A inhibitor is CPI-455.

8. The method of claim 1, wherein the KDM5A inhibitor is administered in an amount ranging from about 0.01 mg to 10 mg.

9. The method of claim 2, wherein the subject has ovarian cancer.

10. The method of claim 2, wherein the subject has high-grade serous ovarian cancer (HGSOC).

11. The method of claim 2, wherein the subject has triple negative breast cancer.

12. The method of claim 2, wherein the KDM5A inhibitor is CPI-455.

13. The method of claim 2, wherein the KDM5A inhibitor is administered in an amount ranging from about 0.01 mg to 10 mg.

14. The method of claim 3, wherein the cancer is ovarian cancer.

15. The method of claim 3, wherein the cancer is high-grade serous ovarian cancer (HGSOC).

16. The method of claim 3, wherein the cancer is triple negative breast cancer.

17. The method of claim 3, wherein the KDM5A inhibitor is CPI-455.

18. The method of claim 3, wherein the KDM5A inhibitor is administered in an amount ranging from about 0.01 mg to 10 mg.

19. The method of claim 3, comprising reducing the level of KDM5A mRNA in the subject.

20. The method of claim 19, wherein the KDM5A mRNA levels are reduced using siRNA.


SEQUENCE LISTING (2 sequences)

SEQ ID NO: 1 - DNA, 21 bp, synthetic construct
  source 1..21: mol_type = other DNA
cctttgagtgacttagaggaa

SEQ ID NO: 2 - DNA, 21 bp, synthetic construct
  source 1..21: mol_type = other DNA
caaagagaacaagacgagtta